(19) United States
(12) Patent Application Publication
(10) Pub. No.: US 2022/0185756 A1
(43) Pub. Date: Jun. 16, 2022

(54) New Industrial Process for Manufacturing of Perfluoro (Methyl Vinyl Ether)(PFMVE) and of 1,1,2,2-Tetrafluoro-1-(Trifluoromethoxy)ethane (TFTFME)

(71) Applicant: Fujian Yongjing Technology Co.,Ltd, Shaowu (CN)

(72) Inventors: WEIFEN LUO, Shaowu (CN); LVZHOU QIU, Shaowu (CN)

(73) Assignee: Fujian Yongjing Technology Co.,Ltd, Shaowu (CN)

(21) Appl. No.: 17/565,494

(22) Filed: Dec. 30, 2021

Related U.S. Application Data
(63) Continuation of application No. PCT/CN2021/120887, filed on Sep. 29, 2021.

(30) Foreign Application Priority Data
Nov. 25, 2020 (DE) 102020131159.1

(51) Int. Cl. C07C 41/22; B01J 19/18; C07C 41/20; B01J 19/00

(52) U.S. Cl. CPC C07C 41/22; B01J 19/1868; C07C 41/20; B01J 2219/00162; B01J 19/0053; B01J 2219/00051; B01J 19/0013

(57) ABSTRACT

The invention relates to a new industrial process for manufacturing of perfluoro(methylvinylether) (PFMVE), and of 1,1,2,2-tetrafluoro-1-(trifluoromethoxy)ethane (TFTFME) (E 227), involving reactions in liquid phase and performing reactions, for example, in a (closed) column reactor or in a microreactor, respectively. The invention also relates to a new industrial process for manufacturing of perfluoro(methyl vinyl ether) (PFMVE) by HF-elimination from the compound 1,1,2,2-tetrafluoro-1-(trifluoromethoxy)ethane (TFTFME) (E 227). The invention also relates to a new industrial process for manufacturing of the compound 1,1,2,2-tetrafluoro-1-(trifluoromethoxy)ethane (TFTFME) (E 227) by selective fluorination of the compound HFE-254 (1,1,2,2-tetrafluoro-1-(methoxy)ethane), i.e., perfluorination of only the CH₃O-group (i.e., methoxy-group) of the compound HFE-254 is selectively fluorinated to a CF₃O-group (i.e., trifluoromethoxy-group).

### BACKGROUND OF THE INVENTION

### 1. Field of the Invention

The invention relates to a new industrial process for manufacturing of perfluoro(methylvinylether) (PFMVE), and of 1,1,2,2-tetrafluoro-1-(trifluoromethoxy)ethane (TFTFME) (E 227). The invention also relates to a new industrial process for manufacturing of perfluoro(methyl vinyl ether) (PFMVE) out of 1,1,2,2-tetrafluoro-1-(trifluoromethoxy)ethane (TFTFME) (E 227).

### 2. Description of the Prior Art

The compound perfluoro(methyl vinyl ether) (PFMVE), also named perfluoromethoxyethene (IUPAC) or perfluoromethoxyethylene, and the compound 1,1,2,2-tetrafluoro-1-(trifluoromethoxy)ethane (TFTFME) (E 227) are known in the state of the art. The compound perfluoro(methyl vinyl ether) (PFMVE) is a halogenated derivative of methoxyethene (H₃CO—CH═CH₂; CAS number: 107-25-5; other names are ethenyl methyl ether or vinyl methyl ether, but the preferred IUPAC name is methoxyethene), which in turn is a derivative of ethylene (IUPAC name: ethene; H₂C═CH₂; CAS number: 74-85-1).

The compound 1,2,2-tetrafluoro-1-(trifluoromethoxy)ethane (TFTFME) (E 227) commonly is also named: trifluoromethyl-1H-pentafluoroethyl ether, 2-hydryl-F-ethyl F-methyl ether, 1,1,2,2-tetrafluoroethyltrifluoromethyl ether; perfluoro 2H-ethyl methyl ether, 1-trifluoromethoxy-1,1,2,2-tetrafluoroethane, or CF₃OCF₂CHF₂.

The compound perfluoro(methyl vinyl ether) (PFMVE) can be prepared also via or starting from the compound 2-fluoro-1,2-dichloro-trifluoromethoxyethylene (FCTFE), which is also named 2-fluoro-1,2-dichloro-trifluoromethyl-vinylether or 2-fluoro-1,2-dichloro-trifluoromethoxyethene (IUPAC), which compound and the preparation thereof is also known in the state of the art.

Perfluoro(methyl vinyl ether), for example, is a monomer used to make some fluoroelastomers.

The synthesis of these compounds, perfluoro(methyl vinyl ether) (PFMVE) and of 1,1,2,2-tetrafluoro-1-(trifluoromethoxy)ethane (TFTFME) (E 227), having the following formulae (I) and (II), is also known in the state of the art, as well as the synthesis of the compound 2-fluoro-1,2-dichloro-trifluoromethoxyethylene (FCTFE), see formula (IV), is known in the state of the art.

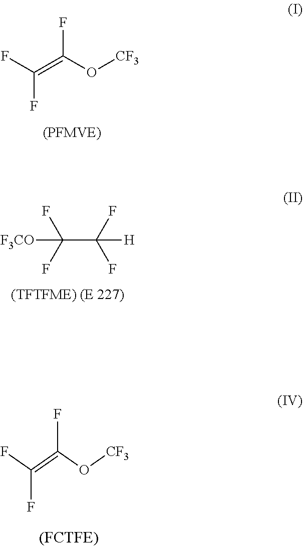 [SMILES: FC(F)=C(F)OC(F)(F)F ; FC(F)C(F)(F)OC(F)(F)F ; FC(F)=C(F)OC(F)(F)F]

However, the known syntheses, as exemplified herein after, of the compounds perfluoro(methyl vinyl ether) (PFMVE), 1,1,2,2-tetrafluoro-1-(trifluoromethoxy)ethane (TFTFME) (E 227), and of the PFMVE-precursor compound 2-fluoro-1,2-dichloro-trifluoromethoxyethylene (FCTFE), have disadvantages, and there is a desire to provide improved processes of manufacturing especially the said compounds perfluoro(methyl vinyl ether) (PFMVE) and 1,1,2,2-tetrafluoro-1-(trifluoromethoxy)ethane (TFTFME) (E 227), respectively.

In early days, Du Pont in U.S. Pat. No. 3,180,895 (1965) disclosed a process for PFMVE out of reaction of hexafluoropropylene oxide with acid fluorides followed by decarboxylation according to:

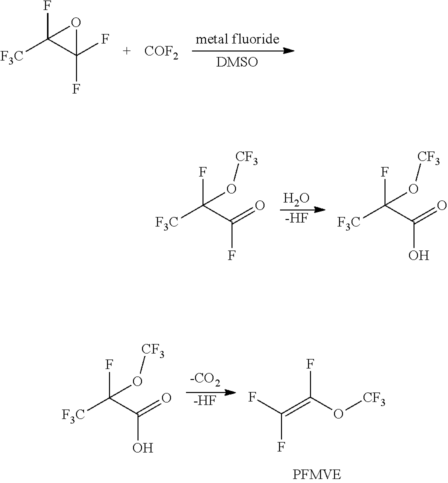

This route is quite complicated regarding handling, safety and availability of raw materials. Especially starting with toxic gaseous raw materials followed by liquid intermediates and intermediates in salt form (for decarboxylation usually the salt is preferred) which ends again in a gas is very challenging. Besides handling, lot of amounts of toxic waste water and toxic side materials are produced and causes environmental drawbacks.

A modification and improvement also described already by Du Pont is the direct usage of the 2-perfluoromethoxy-propionylfluoride over dry potassium sulphate pellets at 300° C. As this is no catalytic process the potassium sulphate cannot be recycled. Both procedures do not fit for large industrial scale.

Alternatively ZhonglanChengung Chemical in CN1318366 (2005) disclosed a preparation of PFVME out of 1,2-dichloro-1,1,2-trifluoro-2-(trifluoromethoxy)ethane.

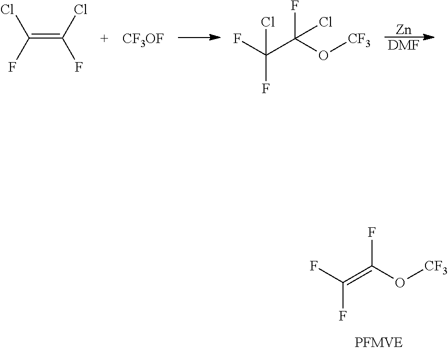

Another route is described by SinochemLantian which includes the pyrolysis of the 2-perfluoromethoxy-propionylfluoride in a fluidized bed in CN107814689 (2018). In another application, SinochemLantian in CN105367392 discloses the usage of CF₃O-ammonium salt and it's reaction with chlorotrifluoroethylene but after reaction the work up is complicated, recycling of formed ammonium salts are not possible.

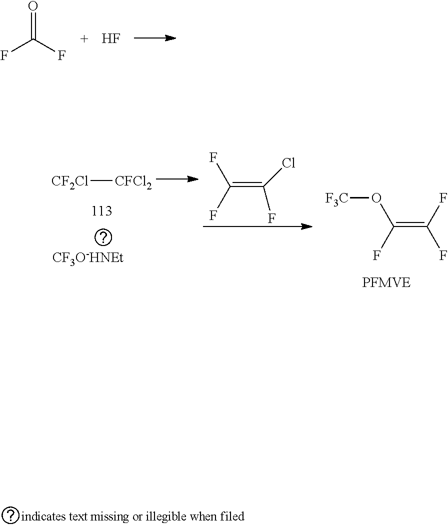

Other known methods for preparing hydrogen containing derivatives are also quite complicated. For example, the trifluoromethoxy vinylether is disclosed in U.S. Pat. No. 3,162,622 (1994, Du Pont). For this compound, which is technically much easier to prepare than the perfluoro(methylvinylether (PFVME), Du Pont disclosed a process starting from halogeno-trifluoromethylvinyl ether by treatment with base. The starting material 2-chloro- or 2-bromo-trifluoromethyl-ether is prepared by a three step process starting with reaction of 2-halogenothanol and carbonyl fluoride to give an intermediate which is finally fluorinated to the 2-halogeno-trifluoromethyl-vinyl-ether with SF4, here an example outlined for 2-chloroethanol.

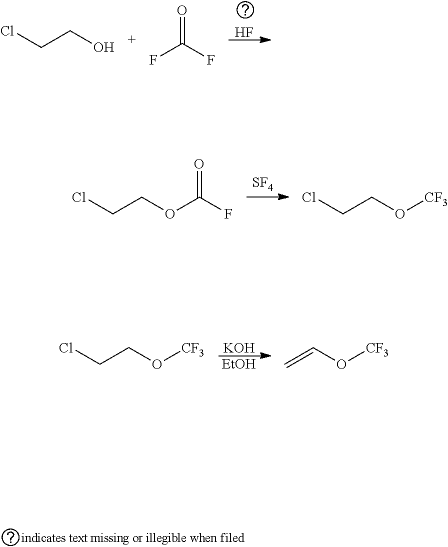

Other methods to preparetrifluoromethoxy vinylethers are disclosed by Kamil et al. in Inorganic Chemistry (1986), 25(3), 376-80, wherein trifluoromethyl hypochlorite in a 1,2-addition reaction is converted with halogenated olefins to the corresponding halogenated trifluoromethoxy halogenoalkane followed by H-Hal elimination:

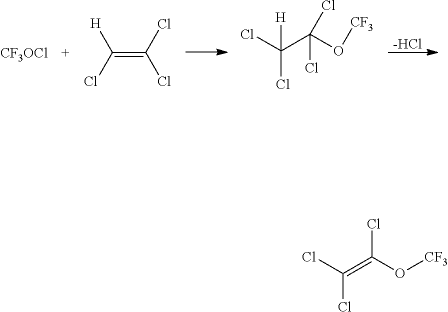 [SMILES: "CF3OCl" ; ClC=C(Cl)Cl ; FC(F)(F)OC(Cl)(Cl)C(Cl)Cl ; "-HCl" ; FC(F)(F)OC(Cl)=C(Cl)Cl]

The CF₃OCl (trifluoromethyl hypochlorite) is known to be prepared by reaction of carbonyl fluoride and CF like disclosed in DE1953144 (1969). Solvay Specialty Polymers In EP1801091 (2007) Solvay Specialty Polymers discloses the addition of CF₃OF to trichloroethylene in a stirred vessel and this same reaction but using a so called microreactor was disclosed many years later in WO2019/110710, with the drawback to be operated at very deep temperature of −50° C., to yield 98% of the 1,2-addition product mixture. This mixture then was treated with tetrabutylammonium hydroxide in aqueous solution to yield 92% FCTFE, but with the disadvantage of much environmental unfriendly salt and waste water formation.

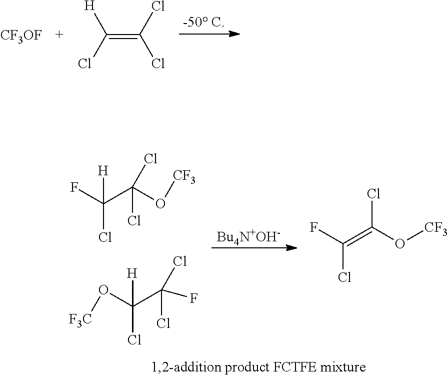

For preparing PFMVE, in an additional step the FCTFE was subjected to an addition of F₂ and a dehydrohalogenation reaction, the latter disclosed also already by Solvay Specialty Polymers in in WO2012/104365.

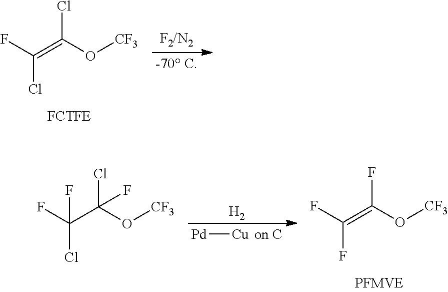

Good selectivity was reported for all steps, but two steps are with deep temperature reactions, one step with waste water and salt formation, and one step in gas phase, and all of these steps have very high energy consumption and might have some economic limitations in industrial scale.

All existing methods in the state of the art to prepare PFMVE involve several challenges which are, for example: (1) handling of gaseous organic fluorinated starting materials and of the gaseous product, and (2) handling of very toxic materials, such as, for example, fluorophosgene (COF₂; also known as carbonyl difluoride), hydrogen fluoride (HF), and hexafluoropropylene epoxide (HFPO), and (3) very strong corrosion activity of reactants against reactor and other equipment.

All existing processes of the state of the art involve at least five chemical steps starting from a fluorine-free starting material like chloroform (CHCl₃) which is, e.g., a starting material for the compound difluorochloromethane (CF₂ClH), which in turn is a starting material for preparing the compound hexafluoropropylene (HFP), and in addition the usage of hardly to prepare fluorophosgene (COF₂; also known as carbonyl difluoride), and reactive and sensitive intermediate compounds such as, e.g., hexafluoropropylene epoxide (HFPO) out of the compound hexafluoropropylene (HFP). All these challenges lead to quite high manufacturing costs, high consumption of energy and much toxic waste formation, e.g., formation of undesired salts and/or undesired organic compounds.

Also, for the compound E 227 (TFTFME), no industrial suitable economic process is known. Hitherto, for example, the compound E 227 (TFTFME) is prepared by addition of hydrogen halide (H-Hal) to the compound PFMVE as disclosed by Gubanov, V. A.; et al. in ZhurnalObshcheiKhimii (1964), 34(8), 2802-3.

As shown herein before the prior art processes are not yet optimal and have several disadvantages. Such disadvantages of the prior art processes, for example, in particular encompass salt formation and high energy consumption. The high energy consumption in the prior art processes, e.g., is due to reaction step sequences requiring cooling in one step (liquid phase reaction step) and heating in another step (gas phase reaction step).

Accordingly, there is a high demand of enabling large-scale and/or industrial production of perfluoro(methyl vinyl ether) (PFMVE), also via the compound 1,1,2,2-tetrafluoro-1-(trifluoromethoxy)ethane (TFTFME) (E 227) which is a suitable intermediate in the manufacture of perfluoro(methyl vinyl ether) (PFMVE), and of the compound 1,1,2,2-tetrafluoro-1-(trifluoromethoxy)ethane (TFTFME) (E 227) itself, respectively, wherein the manufacture of PFMVE and/or TFTFME (E 227) avoids the disadvantages of the prior art processes, and in particular does not encompass salt formation and has particularly less energy consumption than said prior art processes.

Thus, it is an object of the present invention to provide an efficient and simplified new industrial process for manufacturing of perfluoro(methyl vinyl ether) (PFMVE), and/or of and of the compound 1,1,2,2-tetrafluoro-1-(trifluoromethoxy)ethane (TFTFME) (E 227), respectively, which may serve as an intermediate compound in the manufacture of said perfluoro(methyl vinyl ether) (PFMVE).

It is a further object of the present invention to also provide an efficient and simplified new industrial process for manufacturing of perfluoro(methyl vinyl ether) (PFMVE) via or out of the compound 1,1,2,2-tetrafluoro-1-(trifluoromethoxy)ethane (TFTFME) (E 227).

It is preferably another object of the present invention to provide an efficient and simplified new industrial process for manufacturing of perfluoro(methyl vinyl ether) (PFMVE), and/or of the compound 1,1,2,2-tetrafluoro-1-(trifluoromethoxy)ethane (TFTFME) (E 227), and preferably enabling large-scale and/or industrial production of PFMVE and/or of TFTFME (E 227) by means of special equipment and special reactor design.

### SUMMARY OF THE INVENTION

The object of the invention is solved as defined in the claims, and described herein after in detail.

FIG. 1 shows the manufacture of PFVME out of HFE-254 (1,1,2,2-tetrafluoro-1-(methoxy)ethane) and F₂-fluorination gas, over E 227 (TFTFME) as an intermediate compound, and using a counter current reactor system (e.g., a gas scrubber system).

Two step batch process in a counter-current system. See, for example, reaction Scheme 3 below and Example 1. The reservoir is containing the liquid raw material HFE-254 (1,1,2,2-tetrafluoro-1-(methoxy)ethane) for the first step. The F₂-fluorination gas feed is introduced in a first step for performing a fluorination (A) reaction as described below, and to obtain fluorination product E 227 (TFTFME) as an intermediate compound. In a second step (not shown) the intermediate fluorination product E 227 (TFTFME) compound is subjected to an HF-elimination (B) reaction to yield the product PFVME which is collected as raw product in a trap. The HF formed in the HF-elimination (B) reaction (second step) leaves as purge gas during second step reaction together with inert gas used for purging the reactor system as described herein. In this Example 1, the HF-elimination (B) reaction step is performed as a base induced elimination using NEt₃ (triethylamine) as the organic base. If the second step is not performed, then the fluorination product E 227 (TFTFME) compound is the final product and can be isolated and/or purified, for example as shown in Example 3. The reactor design shown in FIG. 1 (one or more packed bed towers) is representative for performing reactions in a counter-current reactor system, in particular in a loop reactor system, or in a counter-current (loop) system (“inverse gas scrubber system”).

FIG. 2 shows the manufacture of PFMVE by reaction of HFE-254 (1,1,2,2-tetrafluoro-1-(methoxy)ethane) with F₂-fluorination gas, over E 227 (TFTFME) as an intermediate compound in a sequence of two microreactors.

The first microreactor is a SiC-microreactor for fluorination (A) reaction, and the second microreactor is a Ni-microreactor for HF-elimination (B) reaction. See, for example, reaction Scheme 3 below and Example 4. The reservoir is containing the liquid raw material HFE-254 (1,1,2,2-tetrafluoro-1-(methoxy)ethane) for the first step. The F₂-fluorination gas feed is introduced in a first step for performing a fluorination (A) reaction as described below, and to obtain fluorination product E 227 (TFTFME) as an intermediate compound. The fluorination product E 227 (TFTFME) and the HF formed in the first fluorination step (A) are collected in a buffer tank, and the inert gas (e.g., N₂) leaves as purge gas. In a second step the intermediate fluorination product E 227 (TFTFME) compound is subjected to an HF-elimination (B) reaction to yield the product PFVME which is collected as raw product in a trap together with the HF formed in the HF-elimination (B) reaction (second step) as described herein. In this Example 4, the HF-elimination (B) reaction step is performed as a catalytic thermal elimination step. The catalysis is on the Ni (nickel) contained in the microreactor, as described herein below. If the second step is not performed, then the fluorination product E 227 (TFTFME) compound is the final product and can be isolated and/or purified, for example by distillation as shown in Example 3. The reactor design shown in FIG. 2 (one or more microreactors) is representative for performing reactions in a tube reactor system, a continuous flow reactor system, in a coil reactor system or in a microreactor system.

Surprisingly, now it has been found that the compound perfluoro(methyl vinyl ether) (PFMVE) of formula (I) can easily be prepared by following, in principle, the reaction Scheme 1, avoiding hazardous gaseous compound such like in particular fluorophosgene (COF₂; also known as carbonyl difluoride). Especially, this is achieved by the process of the present invention in that it is based on the use of the compound 1,1,2,2-tetrafluoro-1-(methoxy)ethane (HFE-254) as the (initial) starting compound, e.g. in the manufacture of the intermediate compound 1,1,2,2-tetrafluoro-1-(trifluoromethoxy)ethane (TFTFME) (E 227) of formula (II), which in turn can be also a starting compound for the manufacture of the compound perfluoro(methyl vinyl ether) (PFMVE) of formula (I). The preparation of the compound 1,1,2,2-tetrafluoro-1-(methoxy)ethane (HFE-254) is known in the state of the art, e.g., in particular by the addition of methanol (CH₃—OH) to the compound tetrafluoroethylene (TFE) as shown in the first reaction of reaction Scheme 2, further below. The (initial) compound 1,1,2,2-tetrafluoro-1-(methoxy)ethane (HFE-254) (CAS number: 425-88-7) is also known, for example, under the following names (synonyms) or common terminology: 1,1,2,2-tetrafluoro-1-methoxyethane (1,1,2,2-tetrafluoro-1-methoxy-ethane); 1-methoxy-1,1,2,2-tetrafluoroethane; 1,1,2,2-tetrafluoroethyl methyl ether (1,1,2,2-tetrafluoroethylmethylether); methyl 1,1,2,2-tetrafluoroethyl ether (methyl-1,1,2,2-tetrafluoroethylether); methyl 1,1,2,2-tetrafluoroethyl (7CI,8CI) ether; 1,1,2,2-tetrafluoroethyl methyl ether; HFE-254; HFE-254CB1; HFE-254cb2; HFE-254pc; C₃H₄F₄O. The (initial) starting compound 1,1,2,2-tetrafluoro-1-(methoxy)ethane (HFE-254cb2) has a molecular weight of 132.057 g/mol; a density of 1.2939 g/cm³ (at 20° C.); a boiling point of 36.5° C. (at 760 mmHg); and a melting point of −107° C.

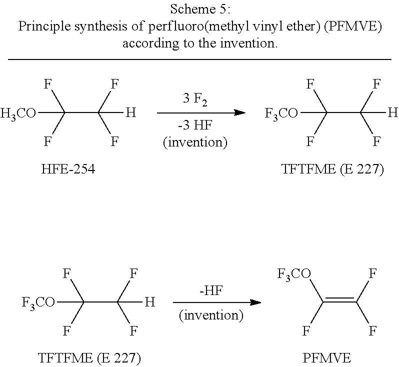

The invention relates also to a new industrial process for manufacturing of perfluoro(methyl vinyl ether) (PFMVE), and/or of the compound 1,1,2,2-tetrafluoro-1-(trifluoromethoxy)ethane (TFTFME) (E 227), respectively, which is a suitable intermediate in the manufacture of perfluoro(methyl vinyl ether) (PFMVE), involving reactions in liquid phase and, for example, performing reactions in a counter-current reactor system, in particular in a loop reactor system, or in a counter-current (loop) system (“inverse gas scrubber system”), as well as in a tube reactor system, a continuous flow reactor system, in a coil reactor system or in a microreactor system, preferably performing reactions in a counter-current reactor system or in a microreactor, respectively, as each described here under and in the claims.

Accordingly, the present invention, as described hereunder in more detail and as defined in the claims, in one aspect relates to a new process for the industrial synthesis of perfluoro(methyl vinylether) (PFMVE) out of the compound HFE-254 (1,1,2,2-tetrafluoro-1-(methoxy)ethane) over E 227 (TFTFME) as an intermediate compound, or directly starting from the compound E 227 (TFTFME). In particular, in a preferred aspect of the invention, the new process for the industrial synthesis of perfluoro(methyl vinyl ether) (PFMVE) includes a selective direct fluorination step with elemental fluorine (F₂) of the compound HFE-254 (1,1,2,2-tetrafluoro-1-(methoxy)ethane) used as the initial starting material. Here, in another aspect the invention also relates to a new process for the industrial synthesis the compound E 227 (TFTFME) as final product compound out of the compound HFE-254 (1,1,2,2-tetrafluoro-1-(methoxy)ethane).

The compound E 227 (TFTFME) can be particularly used as an environmentally friendly starting material in the new process for the industrial synthesis of perfluoro(methyl vinyl ether) (PFMVE), either as an intermediate E 227 (TFTFME) compound when starting from the compound HFE-254 (1,1,2,2-tetrafluoro-1-(methoxy)ethane), or directly as the E 227 (TFTFME) starting material compound in said synthesis of perfluoro(methyl vinyl ether) (PFMVE).

The compound 1,1,2,2-tetrafluoro-1-(trifluoromethoxy)ethane (TFTFME) (E 227) (CAS number: 2356-61-8) has a molecular weight of 186.028 g/mol, a density of 1.512 g/cm³, and a boiling point of −12.105° C. (at 760 mmHg).

Furthermore, the isolated, and optionally purified, compound E 227 (TFTFME) can be used as environmentally friendly CFC-12 refrigerant alternative, as disclosed by S. Devotta in International Journal of Refrigeration (1993), 16(2), 84-90, and in WO9711138, and it can be potentially used as solvent in polymerization reactions like disclosed by Daikin in JP2012241128. Also usage of the compound E 227 (TFTFME) as etching gas was disclosed by Daikin in JPH11124352.

In JPH11124352 Daikin proposes a method for producing fluorinated ether, hardly producing by-product, in high conversion. The method for producing fluorinated ether comprises subjecting 1,1,2,2-tetrafluoroethylmethyl ether to contact reaction with fluorine gas in the presence of a hydrogen fluoride or a solvent, other than hydrogen fluoride, inert to fluorine gas or in the state diluted with a gas which is inert to the fluorine gas in vapour phase. For example, the Daikin describes a fluorination process in chlorotrifluoroethylene oil, but the process does not take place in reactor systems and not under conditions such as are used in the context of the present invention such as, for example, a counter-current reactor system, in particular a loop reactor system, or a counter-current (loop) system (“inverse gas scrubber system”), as well as a tube reactor system, a continuous flow reactor system, a coil reactor system or a microreactor system, preferably a counter-current reactor system or a microreactor, respectively, as each described here under and in the claims. In addition, however, the process described by Daikin in JPH11124352 yields the compound E 227 (TFTFME) only with a very low yield of 2.57%, next to other products. For example compounds, useful as dry-etching gases (no data), are prepared by either of the following method: (1) reaction of HCF₂CF₂OMe with fluorine (F₂) in the presence of HF or (2) gas-phase reaction of HCF₂CF₂OMe with fluorine (F₂) in the presence of gases inert to fluorine (F₂). For example, in JPH11124352 (Daikin) the compound HCF₂CF₂OCH₃ (E 227) (TFTFME) was fluorinated by fluorine (F₂) in chlorotrifluoroethylene oligomer oil as the solvent at room temperature to give HCF₂OCF₂CF₃, CF₃OCF₂CHF₂ (i.e., compound E 227, TFTFME), CH₂FOCF₂CF₃, HCF₂OCF₂CHF₂, and FCH₂OCF₂CHF₂ with 9.27%, 2.57% (i.e., compound E 227, TFTFME), 6.5%, 50.56%, and 31.1% selectivity, resp., at 98% conversion.

Daikin in JPH11124352 proposes a method for producing fluorinated ether, hardly producing by-product, in high conversion. The method for producing fluorinated ether comprises subjecting 1,1,2,2-tetrafluoroethylmethyl ether to contact reaction with fluorine gas in the presence of a hydrogen fluoride or a solvent, other than hydrogen fluoride, inert to fluorine gas or in the state diluted with a gas which is inert to the fluorine gas in vapor phase. Das Verfahren bei Daikin beschreibt eine Fluorierung in ChlortrifluorethyleneOil, findet jedoch nicht in Reaktorsystemen und Bedingungen statt, wie diese im Rahmen der vorliegenden Erfindung zur Anwendung kommen, beispielsweise insbesondere nicht in einem Umlaufreaktor oder in einem Mikroreaktor. Zudem erzielt das Verfahren erzielt jedoch lediglich die Verbindung E 227 (TFTFME) mit 2,57% Ausbeute.

However, for the compound E 227 (TFTFME), no industrial suitable economic process is known. Hitherto, for example, the compound E 227 (TFTFME) is prepared by addition of hydrogen halide (H-Hal) to the compound PFMVE as disclosed by Gubanov, V. A.; et al. in ZhurnalObshcheiKhimii (1964), 34(8), 2802-3.

The compound HFE-254(1,1,2,2-tetrafluoro-1-(methoxy)ethane) is a very common hydrofluoroether and it is used in large commercial scale as foam blowing agent like, e.g., described in CN110343227, or it is used as an additive to the electrolyte in Li-Ion batteries (see JP2019135730). The compound HFE-254 is also used as starting material for the synthesis of difluoroacetylfluoride (DFAF) like disclosed in JP2011073984. The DFAF is a key raw material for a pyrazole based fungizide family like BASF's Fluxopyroxad®, Syngenta's Isoprazam®, and Bayer Cropscience's Bixafen®, each having a CF₂H-group at side chain in common. The compound HFE-254 (1,1,2,2-tetrafluoro-1-(methoxy)ethane) is known in the prior art to be prepared by many companies by addition of methanol (CH₃OH) to tetrafluoroethylene (TFE), e.g., like disclosed in CN103772156 and RU2203881. See, for example, reaction Scheme 2, which shows the particularly preferred synthesis method of providing the (initial) starting compound HFE-254 (1,1,2,2-tetrafluoro-1-(methoxy)ethane) for performing the process of the present invention.

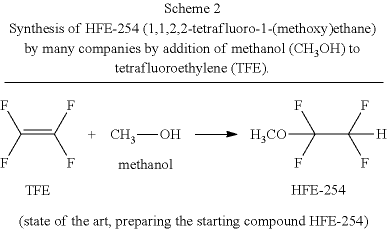

The present invention circumvents the mentioned disadvantages of salt formation and high energy consumption. For example, the present invention circumvents the mentioned disadvantages of the prior art processes, for example, the disadvantages of salt formation and high energy consumption. The high energy consumption in the prior art processes, e.g., is due to reaction step sequences requiring cooling in one step (liquid phase reaction step) and heating in another step (gas phase reaction step).

In contrast to the prior art processes, by exemplification but not intended to be limited to this example, the reaction step sequences according to the present invention avoid such undesired salt formation and undesired high energy consumption, for example (representatively), by using conveniently commercially available compound HFE-254 (1,1,2,2-tetrafluoro-1-(methoxy)ethane), and by direct fluorination, in particular selective direct fluorination, of the methoxy group thereof to a trifluoromethoxy group according to the reaction sequence as shown in Scheme 1 above.

Surprisingly, according to the invention it was found that the direct fluorination, in particular the selective direct fluorination, of the methoxy group (CH₃—O-group) of the compound HFE-254 (1,1,2,2-tetrafluoro-1-(methoxy)ethane), is substantially not or only very little attacking the CF₂H-group in second position formed during the fluorination in the resulting (intermediate) compound 1,1,2,2-tetrafluoro-1-(trifluoromethoxy)ethane (TFTFME) (E 227). The said CF₂H-group is strongly deactivated by the two fluorine atoms which are strongly electron withdrawing and thus preserve the hydrogen (H) in the said CF₂H-group. On the other hand, surprisingly the oxygen atom of the methoxy group (CH₃—O-group) is strongly activating the directly connected methyl group (CH₃-group) thereof, to promote the perfluorination of the methoxy group (CH₃—O-group), and thus directly yield the desired trifluoromethoxy group (CF₃—O-group); therefore, surprisingly the fluorination does not stop at an intermediate O—CF₂H—O-group, even despite this intermediate group already contains two deactivating fluorine atoms. Thus, in the compound HFE-254 (1,1,2,2-tetrafluoro-1-(methoxy)ethane), only the CH₃O-group (i.e., methoxy-group) of the compound is selectively fluorinated to a CF₃O-group (i.e., trifluoromethoxy-group).

The present invention also provides a selective direct fluorination process (A) for the manufacture or preparation of the compound 1,1,2,2-tetrafluoro-1-(trifluoromethoxy)-ethane (TFTFME) (E 227), as final product compound and/or intermediate compound, by selective direct fluorination of the 1,1,2,2-tetrafluoro-1-(methoxy)ethane, in particular by means of special equipment and special reactor design, for example, as shown in FIG. 1 and FIG. 2, respectively, and as further described hereunder. The special equipment and special reactor design employed by the invention may comprise one or more packed bed towers, e.g., in the form of a gas scrubber system, or one or more microreactors.

It has been discovered that despite the exothermic character of the direct fluorination reaction, e.g., within a given time period (e.g., less than 10 hours, or even less than 5 hours), the reaction of the invention can be performed as a larger scale reaction with high conversion rates, and without major impurities in the resulting fluorinated product. The fluorinated product can be produced in kilogram scale quantities, e.g., the direct fluorination process of the invention can be performed in a large-scale and/or industrial production of a fluorinated inorganic compound or fluorinated organic compound, respectively.

Particular examples of performing the process of the present invention are described in the context of the FIG. 1 (closed column reactor system) and FIG. 2 (microreactor system).

The direct fluorination (A) process and the HF-elimination (B) process can be performed independently and separately of each other, either yielding the fluorination product compound E 227 (TFTFME) or yielding the HF-elimination product compound PFVME.

Alternatively, the direct fluorination (A) process and the HF-elimination (B) process can be performed subsequently as a two-step process with or without isolating and/or purifying the intermediate fluorination product compound E 227 (TFTFME), and to finally yield the HF-elimination product compound PFVME.

If a microreactor system is used, as e.g. shown in FIG. 2, the fluorination product compound E 227 (TFTFME) and the HF formed in the first fluorination step (A), preferably are collected in a buffer tank, and the inert gas (e.g., N₂) leaves as purge gas. The compound E 227 (TFTFME) and the HF, together collected in said buffer tank, if desired can be separated from each other by distillation. Thereafter, the compound E 227 (TFTFME), if desired with or without further purification, is transferred into the second microreactor to perform the HF-elimination (B) process to finally yield the HF-elimination product compound PFVME.

Alternatively, if the direct fluorination (A) process and the HF-elimination (B) process performed subsequently as a two-step process to finally yield the HF-elimination product compound PFVME, it is not obligatory to separate off the HF formed in the direct fluorination (A) process, and the fluorination product compound E 227 (TFTFME) and the HF formed in the first fluorination step (A), preferably are collected in a buffer tank, and the inert gas (e.g., N₂) leaves as purge gas, and then the intermediate fluorination product compound E 227 (TFTFME) and the HF together are transferred into the second microreactor to perform the HF-elimination (B) process to finally yield the HF-elimination product compound PFVME. In this case, it is necessary to separate off the HF formed in the two subsequent process steps of direct fluorination (A) and the HF-elimination (B) only once, after yielding the final HF-elimination product compound PFVME. Separating off the HF formed in the said two subsequent process steps (A) and (B) can be performed, for example, by distillation. Alternatively, separating off the HF formed in the said two subsequent process steps (A) and (B) can be performed, for example, by using preferably an organic base as described herein and more preferably by using an organic base such as, for example, NEt₃ and NBu₃.

Direct Fluorination (A):

The term “direct fluorination” means introducing one or more fluorine atoms into a compound by chemically reacting a starting compound, e.g. according to the present invention in the compound HFE-254 (1,1,2,2-tetrafluoro-1-(methoxy)ethane), with elemental fluorine (F₂) such that one or more fluorine atoms are covalently bound into the said compound, thus replacing one or more hydrogen atoms therein. The term “selective direct fluorination” means introducing fluorine atoms only into the methoxy-group (CH₃O-group) of the said compound.

Accordingly, the direct fluorination of the present invention provides a high efficient process for the manufacture or for preparation of the compound 1,1,2,2-tetrafluoro-1-(trifluoromethoxy)ethane (TFTFME) (E 227), as final product compound and/or intermediate compound, by selective direct fluorination of the 1,1,2,2-tetrafluoro-1-(methoxy)ethane, using fluorine gas (F₂), herein also termed “F₂-fluorination gas”.

The F₂-fluorination gas used in the invention can be of any origin. For example, the present invention can also use a F₂-fluorination gas in the selective direct fluorination step (A) using fluorine gas (F₂), as it comes directly out (e.g., without further purification) of an F₂-electrolysis reactor (fluorine cells), and optionally is only diluted by an inert gas (or mixture thereof) to a desired fluorine (F₂) concentration. Of course, the fluorine gas (F₂) coming from an F₂-electrolysis reactor (fluorine cells), if desired, can also be subjected to purification before it is used in the selective direct fluorination step (A); optionally this purified fluorine gas (F₂) originally derived from an F₂-electrolysis reactor (fluorine cells) is only diluted to some extent by an inert gas (or mixture thereof) to a desired fluorine (F₂) concentration.

Purification of the fluorination gas as it is derived from an F₂-electrolysis reactor (fluorine cell), if desired, optionally is possible, to remove a part or all by-products and traces formed in the F₂-electrolysis reactor (fluorine cell), prior to its use as fluorination gas in the process of the present invention. However, in the process of the present invention such a partial or complete purification is not obligatory, and the fluorination gas can be directly used as it comes out of an F₂-electrolysis reactor (fluorine cell), but if desired, optionally is only diluted by inert gas to a desired fluorine (F₂) concentration. When employing an F₂-fluorination gas derived from an F₂-electrolysis reactor (fluorine cell), purified or unpurified, thus it can be diluted by an inert gas, most preferably by nitrogen (N₂), to the extent desired.

The fluorine (F₂) concentration in the F₂-fluorination gas may vary in wide range, for example, of from about 1% by volume of elemental fluorine (F₂) up to about almost 100% by volume of elemental fluorine (F₂), based on the total F₂-fluorination gas composition as 100% by volume. The term “about almost 100% by volume of elemental fluorine (F₂)” means that for technical reason, e.g., if the elemental fluorine (F₂) is taken from a fluorine cell, technical grade elemental fluorine (F₂) will contain traces of impurities, for example some tetrafluoromethane (CF₄) formed during electrolysis. Hence, the term “about almost 100% by volume of elemental fluorine (F₂)” will be understood by the person skilled in the field, e.g., as up to about 99.9%, up to about 99.8%, up to about 99.7%, up to about 99.6%, up to about 99.5%, or up to about 99%+1%, respectively, each by volume of elemental fluorine (F₂).

Typical ranges of lower fluorine (F₂) concentrations in the F₂-fluorination gas, for example, are of from about 1% by volume of elemental fluorine (F₂) up to about 30% by volume of elemental fluorine (F₂), more preferably of from about 5% by volume of elemental fluorine (F₂) up to about 25% by volume of elemental fluorine (F₂), even more preferably of from about 5% by volume of elemental fluorine (F₂) up to about 20% by volume of elemental fluorine (F₂), each range based on the total F₂-fluorination gas composition as 100% by volume. The lower fluorine (F₂) concentrations in the F₂-fluorination gas, for example, can be applied when performing reactions in a counter-current reactor system, in particular in a loop reactor system, or in a counter-current (loop) system (“inverse gas scrubber system”).

Typical ranges of higher fluorine (F₂) concentrations in the F₂-fluorination gas, for example, are of from about 85% by volume of elemental fluorine (F₂) up to about almost 100% (as defined herein above) by volume of elemental fluorine (F₂), preferably of from about 90% by volume of elemental fluorine (F₂) up to about almost 100% (as defined herein above) by volume of elemental fluorine (F₂), based on the total F₂-fluorination gas composition as 100% by volume. The higher fluorine (F₂) concentrations in the F₂-fluorination gas, for example, are preferably applied when performing reactions in a tube reactor system, in a continuous flow reactor system, in a coil reactor system, or in a microreactor system, preferably in a microreactor system. However, the said higher fluorine (F₂) concentration in the F₂-fluorination gas, for example, can also be applied when performing reactions in in a counter-current reactor system, in particular in a loop reactor system, or in a counter-current (loop) system (“inverse gas scrubber system”).

It goes without saying that a skilled person will understand that within any of the above given ranges any intermediate values and intermediate ranges can be selected, too.

The term “vol.-%” as used herein means “% by volume”. Unless otherwise stated, all percentages (%) as used herein denote “vol.-%” or “% by volume”, respectively.

The term “inert gas” means a gas that does not undergo chemical reactions under a set of given conditions. Typical inert gases include any noble gas, which make up a class of chemical elements with similar properties, and under standard conditions, are all odorless, colorless, monatomic gases with very low chemical reactivity, for example, such like the noble gases are helium (He), neon (Ne) and argon (Ar), or inert gases such as Nitrogen (N₂). Preferably, (purified) argon (Ar) and/or nitrogen (N₂) gases are used as inert gases due to their high natural abundance (78.3% N₂, 1% Ar in air) and low relative cost. The more preferred inert gas in the context of the invention is nitrogen (N₂). The use of mixtures of said inert gases is possible, too.

The extent of diluting fluorine (F₂) gas in an inert gas or mixture thereof, i.e., the fluorine (F₂) concentration of the F₂-fluorination gas used in the fluorination process step (A), can depend on the special equipment and special reactor design used, for example, as shown in FIG. 1 (one or more packed bed towers) being representative for performing reactions in a counter-current reactor system, in particular in a loop reactor system, or in a counter-current (loop) system (“inverse gas scrubber system”), and, for example, as shown in FIG. 2 (one or more microreactors) being representative for performing reactions in a tube reactor system, a continuous flow reactor system, in a coil reactor system, or in a microreactor system.

In particular, the fluorine (F₂) concentration of the F₂-fluorination gas used in the fluorination process step (A) can be different for a reactor design for performing reactions in a counter-current reactor system, for example, as shown in FIG. 1 (one or more packed bed towers) on the one hand, and for a reactor design for performing reactions in a microreactor system, for example, as shown in FIG. 2 (one or more microreactors).

Direct Fluorination (A) in a Column Reactor, e.g., in a Counter-Current Reactor System:

Preferably, in the F₂-fluorination gas the following (F₂) concentration is adjusted when performing reactions in a counter-current reactor system, in particular in a loop reactor system, or in a counter-current (loop) system (“inverse gas scrubber system”).

Regarding F₂ concentration in the F₂-fluorination gas composition it is noted that in case of a counter-current reactor system, in particular in a loop reactor system, or in a counter-current (loop) system (“inverse gas scrubber system”), for example, as shown in the FIG. 1, the direct fluorination (A) process can be worked equally with inert gas diluted F₂ and concentrated F₂, respectively, since inert gas can escape overhead via the pressure control valve, without any problems, for example, without any hotspots etc. in the reactor, which hotspots would reducing selectivity and yield.

Thus, when performing fluorination (A) reactions in a counter-current reactor system, in particular in a loop reactor system, or in a counter-current (loop) system (“inverse gas scrubber system”), the fluorination (A) reactions can be performed over the whole wide range of fluorine (F₂) concentration in the F₂-fluorination gas, as given here before, that is a fluorine (F₂) concentration in the F₂-fluorination of from about 1% by volume of elemental fluorine (F₂) up to about almost 100% by volume of elemental fluorine (F₂), based on the total F₂-fluorination gas composition as 100% by volume. Accordingly, in this case the fluorination (A) reactions can be performed, for example, (i) in the typical ranges of lower fluorine (F₂) concentration in the F₂-fluorination gas as given above, (ii) in the typical ranges of higher fluorine (F₂) concentration in the F₂-fluorination gas as given above, but as well (iii) in the ranges of middle fluorine (F₂) concentration in the F₂-fluorination gas such as, for example, of from about >30% by volume of elemental fluorine (F₂) up to about <85% by volume of elemental fluorine (F₂).

It goes without saying that a skilled person will understand that within any of the above given ranges any intermediate values and intermediate ranges can be selected, too.

Direct Fluorination (A) in a Continuous Flow Reactor System, e.g., Microreactor System:

Preferably, in the F₂-fluorination gas the following fluorine (F₂) concentration is adjusted when performing reactions in a tube reactor system, in a continuous flow reactor system, in a coil reactor system, or in a microreactor system, preferably in a microreactor system.

Regarding F₂ concentration in the F₂-fluorination gas composition it is noted that in case of a tube reactor system, a continuous flow reactor system, a coil reactor system, or a microreactor system, preferably in a microreactor system, the fluorination (A) reaction preferably is performed, within the above mentioned typical ranges of higher fluorine (F₂) concentration in the F₂-fluorination gas. Thus, the higher fluorine (F₂) concentration in the F₂-fluorination gas preferably applied in a tube reactor system, in a continuous flow reactor system, in a coil reactor system, or in a microreactor system, for example, are of from about 85% by volume of elemental fluorine (F₂) up to about almost 100% by volume of elemental fluorine (F₂), preferably of from about 90% by volume of elemental fluorine (F₂) up to about almost 100% by volume of elemental fluorine (F₂), based on the total F₂-fluorination gas composition as 100% by volume.

Furthermore, regarding F₂-concentration in the F₂-fluorination gas composition it is noted that, whereas in case of a counter-current reactor system, in particular in a loop reactor system, or in a counter-current (loop) system (“inverse gas scrubber system”), the direct fluorination (A) process can be worked equally with inert gas diluted F₂ and concentrated F₂, respectively, as already explained above, in contrast, when performing reactions in a tube reactor system, a continuous flow reactor system, in a coil reactor system, or in a microreactor system, it is highly recommendable and preferred to have as little or even (almost) no inert gas in the F₂-fluorination gas composition, as during performing a reaction in said tube reactor system, continuous flow reactor system, coil reactor system, or microreactor system, no gas can escape, i.e., the inert gases are disadvantageous, because they create bubbles in the channels of a microreactor system and thereby hinder the exchange of heat and causing occurrence of hot spots, which then also would reduce selectivity and yield.

Therefore, if before start of reaction in a microreactor system, the system is continuously floated with an inert gas purge, for example, nitrogen (N₂) inert gas purge, the before start of the fluorination (A) reaction in a microreactor system the concentration of inert gas preferably is rapidly reduced once the feeding of raw materials has started, to adjusted the F₂-concentration in the F₂-fluorination gas to the above said ranges of higher fluorine (F₂) concentration in the F₂-fluorination gas. A fast reduction of inert gas feed is essential as inert gas reduces sharply the heat exchange efficiency in the microchannel reactors.

It goes without saying that a skilled person will understand that within any of the above given ranges any intermediate values and intermediate ranges can be selected, too.

HF-Elimination Reaction (B):

Surprisingly, according to the invention it was found that the compound 1,1,2,2-tetrafluoro-1-(trifluoromethoxy)ethane (TFTFME) (E 227) obtained by the direct fluorination, in particular the selective direct fluorination, of the methoxy group of the compound HFE-254 (1,1,2,2-tetrafluoro-1-(methoxy)ethane) in a first reaction step, as an intermediate compound, can be directly further reacted in a second reaction step, i.e., in an HF-elimination, without being isolated and/or purified, to finally yield the compound perfluoro(methyl vinyl ether) (PFMVE). See reaction Scheme 3.

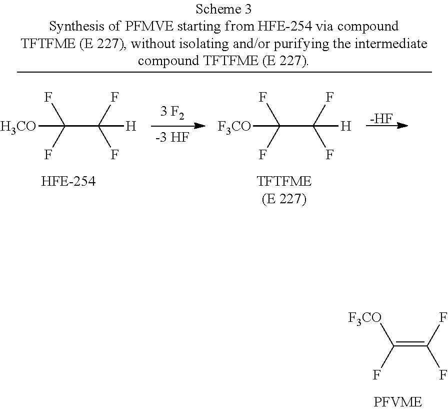

Alternatively, if desired, the compound 1,1,2,2-tetrafluoro-1-(trifluoromethoxy)ethane (TFTFME) (E 227) obtained by the direct fluorination, in particular the selective direct fluorination, of the methoxy group of the compound HFE-254 (1,1,2,2-tetrafluoro-1-(methoxy)ethane) can also be isolated and/or purified, to finally yield the compound 1,1,2,2-tetrafluoro-1-(trifluoromethoxy)ethane (TFTFME) (E 227) as an isolated and/or purified product itself.

Surprisingly, furthermore, for the selective fluorination of the methoxy-group, either a counter-current system can be used in batch or continuously, alternatively a microreactor or coil reactor system can be applied for continuous operation mode.

The HF-elimination step, in a aspect of the invention, can be performed just thermally (non-catalytic), for example by heating above about 100° C. (but this may lead to some undesired polymerization), or the HF-elimination step can be performed as a thermal catalytic HF-elimination by using, for example, Ni (nickel) as the catalyst, for example, Ni (nickel) as the catalyst in a microreactor made out of Nickel or at least comprising an inner Ni-surface or surface with a high Ni (nickel) content, in contact with the 1,1,2,2-tetrafluoro-1-(trifluoromethoxy)ethane (TFTFME) (E 227) starting material.

Alternatively, in another aspect of the invention, the HF-elimination step can be performed as an (exothermic) inorganic and/or organic base induced HF-elimination. Organic base induced HF-elimination is preferred over inorganic base induced HF-elimination, especially if phase separation is desired to isolate and/or purify to finally yield the compound perfluoro(methyl vinyl ether) (PFMVE) as the targeted compound product. Furthermore, organic base induced HF-elimination is preferred over inorganic base induced HF-elimination, especially if a microreactor system is used in the process of the invention, and in particular if the HF-elimination step (B) shall be performed in a microreactor system to finally yield the compound perfluoro(methyl vinyl ether) (PFMVE) as the targeted compound product.

For example, the HF-elimination step can be performed as an (exothermic) organic base induced HF-elimination step, e.g., by using a, preferably water-free, nitrogen-containing base, such like NEt₃ (triethylamine) for performing a, preferably water-free, HF-elimination step.

Furthermore, the HF-elimination step can also be performed as an (exothermic) inorganic base induced, e.g., preferably aqueous, HF-elimination step, e.g., by using an aqueous inorganic base, such like NaOH (sodium hydroxide), KOH (potassium hydroxide) and/or CaCO₃ (calcium carbonate), more preferably as an aqueous, HF-elimination step wherein the inorganic base is a solution in water. Typical inorganic bases that can be used in the present invention are especially NaOH (sodium hydroxide), KOH (potassium hydroxide) and/or CaCO₃ (calcium carbonate), but also LiGH (lithium hydroxide) or NH₄OH ammonium hydroxide can be used. And any combinations thereof can also be used.

The HF-elimination step with an inorganic base can also be performed as an (exothermic) inorganic base induced aqueous HF-elimination step in the in presence or absence of a phase transfer catalyst (PTC). The HF-elimination step preferably is performed as an (exothermic) inorganic base induced aqueous HF-elimination step in the in presence of a phase transfer catalyst (PTC), as this will provide a faster HF-elimination reaction than in the absence of a phase transfer catalyst (PTC) with slow HF-elimination reaction.

The HF-elimination step with an inorganic base can also be performed as an (exothermic) inorganic base induced aqueous HF-elimination step in a counter-current reactor system, for example, in particular in a loop reactor system, a counter-current (loop) system (“inverse gas scrubber system”). The use of inorganic bases in a tube reactor system, a continuous flow reactor system, or in a microreactor system is possible, too, but less preferred, because these tube reactor system, continuous flow reactor system, coil reactor system or microreactor system can get clogged by salt formation and undesired precipitation thereof.

Accordingly, when choosing the HF-elimination step with an inorganic base, the performing of the (exothermic) inorganic base induced aqueous HF-elimination step is preferably done in a counter-current reactor system, for example, in particular in a loop reactor system, a counter-current (loop) system (“inverse gas scrubber system”).

The HF-elimination step as an (exothermic) organic base induced HF-elimination step, e.g., by using a, preferably water-free, nitrogen-containing base, can be performed in any reactor system, for example, in a counter-current reactor system, in particular in a loop reactor system, or in a counter-current (loop) system (“inverse gas scrubber system”), as well as in a tube reactor system, a continuous flow reactor system, coil reactor system or in a microreactor system, respectively.

However, when using a tube reactor system, a continuous flow reactor system, coil reactor system or in a microreactor system, respectively, the HF-elimination step is preferably performed as a thermal catalytic HF-elimination by using, for example, Ni (nickel) as the catalyst in contact with the 1,1,2,2-tetrafluoro-1-(trifluoromethoxy)ethane (E 227) starting material.

For example, Ni (nickel) as the catalyst can be used a tube reactor system, a continuous flow reactor system, or in a microreactor system, respectively, wherein these reactors are made out of Ni (nickel), or the reactor at least comprising an inner Ni-surface or surface with a high Ni (nickel) content for the contact with the 1,1,2,2-tetrafluoro-1-(trifluoromethoxy)ethane (E 227) starting material. Preferably, the Ni (nickel) as the catalyst is used in a microreactor made out of Ni (nickel) or at least comprising an inner Ni-surface or surface with a high Ni (nickel) content for the contact with the 1,1,2,2-tetrafluoro-1-(trifluoromethoxy)ethane (E 227) starting material.

In principle, any organic base can be used in the HF-elimination step according to the invention. However, economically preferred organic bases are selected from the group consisting of NEt₃ (triethylamine), NBu₃ (tributylamine), pyridine, N,N-dimethyl pyridine, DBU (1,8-Diazabicyclo(5.4.0)undec-7-ene), DBN (1,5-Diazabicyclo(4.3.0)non-5-ene); and any derivate thereof; and combinations thereof.

Regarding organic bases, furthermore, these can also be used in “sub-stoichiometric” quantity since organic bases such as, for example, NEt₃ and NBu₃ can absorb more than one equivalent HF, e.g., up to three equivalent HF, for NBu₃ for example up to NBu₃×3HF. The reason is that NEt₃ (triethylamine) and NBu₃ (tributylamine) can form a complex with more than one and up to three hydrogen fluorides (HF).

For example, since three mol of HF formed in the fluorination step (A) have already been removed based on the quantity of one mol HFE-254 after the fluorination step (A), per se only one mol of the organic base such as, for example, NEt₃ and NBu₃, should be necessary in view of (1:1) stoichiometry for the HF-elimination step (B) to remove one mol HF resulting from the HF-elimination step (B). As the organic base such as, for example, NEt₃ and NBu₃, even can take up three HF (complex formation) theoretically only one third (⅓) mol of the organic base such as, for example, NEt₃ and NBu₃, would be necessary instead of one mol organic base such as, for example, NEt₃ and NBu₃, stoichiometry (1:1), and in consideration that each equivalent organic base can take up to three equivalents HF. However, if a phase separation is desired, the organic base such as, for example, NEt₃ and NBu₃, is preferably used in high excess organic base as compared to (1:1) stoichiometry, e.g., in high excess in a range of from about ≥1 mol up to about 1.3 mol of organic base such as, for example, NEt₃ and NBu₃, based on one (1) mol HFE-254 or one (1) mol TFTFME (E 227), respectively; for example, preferably in excess in a range of from about ≥1.1 mol up to about 1.3 mol of organic base, more preferably in excess in a range of from about 1.15 mol up to about 1.3 mol of organic base, even more preferably in excess in a range of from about 1.15 mol up to about 1.25 mol of organic base, most preferably in excess in a range of from about 1.20 mol±0.02 mol of organic base, each based on one (1) mol HFE-254 or one (1) mol TFTFME (E 227), respectively.

If no phase separation is performed, the organic base such as, for example, NEt₃ and NBu₃, is preferably used in minor excess as compared to (1:3) organic base to HF stoichiometry, in consideration that each organic base can take up to three equivalents HF, e.g., in minor excess in a range of from about ≥1% up to about 20% of organic base compared to (1:3) organic base to HF stoichiometry and one (1) mol HFE-254 or one (1) mol TFTFME (E 227), respectively; for example, preferably in excess in a range of from about ≥2%, ≥3%, or ≥4% and each up to about 20% of organic base such as, for example, NEt₃ and NBu₃, more preferably in excess in a range of from 5% up to about 20% of organic base, even more preferably in excess in a range of from 5% up to about 15% of organic base, most preferably in excess in a range of from about 10%±2% of organic base, each based on a (1:3) organic base to HF stoichiometry and one (1) mol HFE-254 or one (1) mol TFTFME (E 227), respectively.

For example, if a quantity of four (4) mol HF is to be consumed, in view of a (1:1) stoichiometry a quantity of four (4) mol of organic base should be necessary for one (1) mol HFE-254 or one (1) mol TFTFME (E 227), respectively. However, in consideration that each equivalent organic base can take up to three equivalents HF, only 4/3 mol (1.333 mol) of organic base are required as compared to (1:3) organic base to HF stoichiometry, and one (1) mol HFE-254 or one (1) mol TFTFME (E 227), respectively. Thus, if a quantity of about 1.5 mol (e.g, 1,467 mol) of organic base such as, for example, NEt₃ and NBu₃, is used for consuming four (4) mol HF, instead of the before calculated 4/3 mol (1.333 mol) of organic base, this corresponds to approximate a 10% excess of organic base. Such exemplified approximate a 10% excess of organic base such as, for example, NEt₃ and NBu₃, e.g., is sufficient in case of no phase separation to be performed.

These organic bases after absorbing HF still stay liquid, and thus are also suitable for a tube reactor system, a continuous flow reactor system, or in a microreactor system, respectively, and especially also for a microreactor system.

Aliphatic organic bases, because of their base strength, are more suitable than hetero aromatic organic bases, and thus aliphatic organic bases will lead to faster HF-elimination reaction, i.e., shorter reaction times, or even possibly to immediate reaction. If pyridine is used, somewhat slower HF-elimination reaction, i.e., longer reaction times, may occur.

Dehydrohalogenation is elimination reaction that eliminates (removes) a hydrogen halide (H-Hal) from a substrate. Hydrogen halides (H-Hal) are known to be diatomic inorganic compounds with the formula H-Hal where “Hal” is one of the halogens, for example, fluorine or chlorine in the context or the present invention. Hydrogen halides, for example, such as in the present invention HF (hydrogen fluoride) is a gas (under the conditions).

Preferably, according to the present invention, the HF-elimination (B) reaction can be performed in a Ni-reactor or in a reactor with a surface with high Ni-content (e.g., a Hastelloy steel) in liquid phase at 100° C. to easily yield the HF-elimination product.

Firstly, having exemplified the invention here before, the process of the present invention, more generally, is directed to a process for the manufacture of PFMVE (perfluoro(methyl vinyl ether)) having the formula (I),

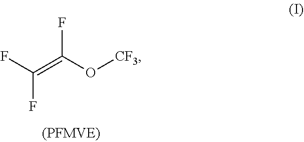

wherein the process comprises the steps of a direct fluorination reaction (A) and HF-elimination reaction (B), in a reactor or reactor system, resistant to elemental fluorine (F₂) and hydrogen fluoride (HF):

(A) in a first reaction step a direct fluorination by reacting the compound HFE-254 (1,1,2,2-tetrafluoro-1-(methoxy)ethane) of formula (III),

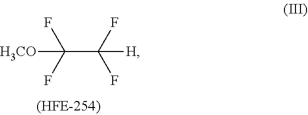

with an about stoichiometric quantity of elemental fluorine (F₂) comprised in a fluorination gas to selectively substitute in the compound of formula (III) the three hydrogen atoms of the 1-(methoxy) group of the compound HFE-254 of formula (III) for fluorine, and wherein the reaction is carried out at temperature in the range of from about 0° C. to about +60° C.

and at a pressure in the range of from about 1 bar absolute bar to about 20 bar absolute

to yield the compound TFTFME (1,1,2,2-tetrafluoro-1-(trifluoromethoxy)ethane), (E 227), of formula (II),

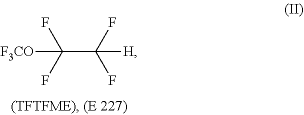

and

with or without isolating and/or purifying the (intermediate) fluorination product (TFTFME), (E 227); preferably without isolating and/or purifying the (intermediate) fluorination product (TFTFME), (E 227),

(B) in a second reaction step an elimination reaction, wherein HF (hydrogen fluoride) is eliminated from the (intermediate) fluorination product (TFTFME), (E 227), of formula (II), obtained in step (A), and the elimination reaction is performed

(i) as a(n) (exothermic) elimination reaction in the presence of one or more nitrogen-containing organic bases,

and/or

in the presence of one or more inorganic bases,

wherein the temperature of the (exothermic) elimination reaction is controlled to not exceed a temperature of about 60° C.,

and wherein the (exothermic) elimination reaction is carried out at a pressure in the range of from about 1 bar absolute bar to about 20 bar absolute

or

(ii) as a, non-catalytic or preferably catalytic, more preferably Ni (nickel) catalytic, thermal elimination reaction at a temperature in the range of about 60° C. to about 120° C.,

to yield the compound PFMVE (perfluoro(methyl vinyl ether)) of formula (I),

and

(C) withdrawing and collecting the compound PFMVE (perfluoro(methyl vinyl ether)) of formula (I) obtained in step (B) from the reactor or reactor system,

and

(D) optionally isolating and/or purifying the compound PFMVE (perfluoro(methyl vinyl ether)) of formula (I).

Secondly, having exemplified the invention here before, the invention also pertains to a process for the manufacture of the compound PFMVE (perfluoro(methyl vinyl ether)) having the formula (I),

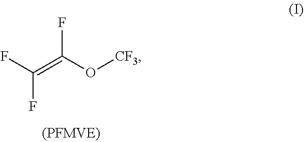

wherein the process comprises, in a reactor or reactor system, resistant to elemental fluorine (F₂) and hydrogen fluoride (HF), performing an HF-elimination reaction step (B), wherein in the elimination reaction, HF (hydrogen fluoride) is eliminated from the compound TFTFME (1,1,2,2-tetrafluoro-1-(trifluoromethoxy)ethane), (E 227), of formula (II),

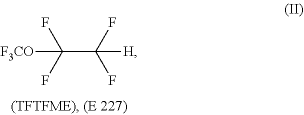

and the elimination reaction step (B) is performed

(i) as a(n) (exothermic) elimination reaction in the presence of one or more nitrogen-containing organic bases,

and/or

in the presence of one or more inorganic bases,

wherein the temperature of the (exothermic) elimination reaction is controlled to not exceed a temperature of about 60° C.,

and wherein the (exothermic) elimination reaction is carried out at a pressure in the range of from about 1 bar absolute bar to about 20 bar absolute,

or

(ii) as a, non-catalytic or preferably catalytic, more preferably Ni (nickel) catalytic, thermal elimination reaction at a temperature in the range of about 60° C. to about 120° C.,

to yield the compound PFMVE (perfluoro(methyl vinyl ether)) of formula (I),

and

(C) withdrawing and collecting the compound PFMVE (perfluoro(methyl vinyl ether)) of formula (I) obtained in step (B) from the reactor or reactor system,

and

(D) optionally isolating and/or purifying the compound PFMVE (perfluoro(methyl vinyl ether)) of formula (I).

Thirdly, having exemplified the invention here before, the invention furthermore pertains to a process for the manufacture of the compound TFTFME (1,1,2,2-tetrafluoro-1-(trifluoromethoxy)ethane), (E 227), of formula (II),

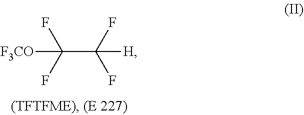

wherein the process comprises, in a reactor or reactor system, resistant to elemental fluorine (F₂) and hydrogen fluoride (HF), performing a direct fluorination reaction step (A), wherein in the direct fluorination reaction the compound HFE-254 (1,1,2,2-tetrafluoro-1-(methoxy)ethane) of formula (III),

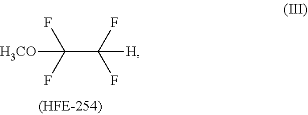

is fluorinated with an about stoichiometric quantity of elemental fluorine (F₂) comprised in a fluorination gas to selectively substitute in the compound of formula (III) the three hydrogen atoms of the 1-(methoxy) group of the compound HFE-254 of formula (III) for fluorine, and

wherein the reaction is carried out at temperature in the range of from about 0° C. to about +60° C. and at a pressure in the range of from about 1 bar absolute bar to about 20 bar absolute,

to yield the compound TFTFME (1,1,2,2-tetrafluoro-1-(trifluoromethoxy)ethane), (E 227), of formula (II),

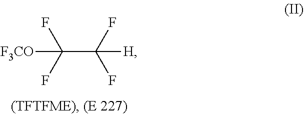

and

(C) withdrawing and collecting the compound TFTFME (1,1,2,2-tetrafluoro-1-(trifluoromethoxy)ethane), (E 227), of formula (II) obtained in step (A) from the reactor or reactor system, and

(D) optionally isolating and/or purifying the compound TFTFME (1,1,2,2-tetrafluoro-1-(trifluoromethoxy)ethane), (E 227), of formula (II).

Thus, in said third aspect, the present invention is directed to a process for the manufacture of compound 1,1,2,2-tetrafluoro-1-(trifluoromethoxy)ethane (TFTFME) (E 227) having the formula (II),

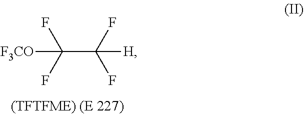

which in the first aspect of the invention is the key intermediate, and in the second aspect of the invention is the key starting material, for the manufacture of the compound PFMVE (perfluoro(methyl vinyl ether)) having the formula (I),

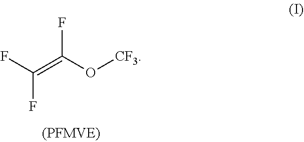

The reaction steps (A) (direct fluorination reaction), and (B) (elimination reaction; H-Hal elimination; H=hydrogen, Hal=halogen atom, i.e., fluorine; hydrogen halogenide elimination, i.e., HF-elimination) in the processes according to the present invention, described herein and in the claims, may be performed in various reactor designs. Example reactor designs include, a loop reactor system, a counter-current (loop) system (“inverse gas scrubber system”), a microreactor system (may include one or more), and coil reactor design. Particular reactor designs are shown in the FIG. 1 (gas scrubber system, counter-current [loop] system), FIG. 2 (microreactor systems). Further, the direct fluorination step in the process of the invention may be performed in a batch or in a continuous manner, respectively. Further, any of the direct fluorination step (A), and the HF-elimination step (B) in the process of the invention may be performed in a batch or in a continuous manner, respectively.

A preferred reactor used in any one of the steps (A) to (B), e.g., in one or more or in all steps of (A) to (B), of the present invention independently is a microreactor system. Preferably, in case of the step (B) (elimination reaction; H-Hal elimination), the reactor is a microreactor system (may include one or more).

Any one of the steps (A) to (B) of the process of the present invention is performed as a liquid phase involving reaction.

In the present invention wherein at least one liquid starting materials is used in the reaction steps (A) to (B), the reactor may be a loop reactor system, a counter-current (loop) system (“inverse gas scrubber system”), but preferably the reactor is microreactor system (may include one or more). See FIG. 1 (gas scrubber system, counter-current [loop] system), or see FIG. 2 (microreactor system), respectively.

In case of a continuous manner process, i.e., when the continuous process according to the invention is performed in any one of the steps (A) to (B), e.g., in one or more or in all steps of (A) to (B), of the present invention independently reactor system is a microreactor system (may include one or more), as described herein and in the claims, and used in continuous operating manner.

In case of a batch manner process, the batch process according to the invention can also be performed in a counter-current system, preferably as described herein and in the claims, in batch operating manner.

The invention also relates to a fluorination process step (A) and/or an HF-elimination step (B), as each described herein and in the claims, optionally either operated in a batch manner or operated in a continuous manner, for the manufacture of the compound perfluoro(methyl vinylether) (PFMVE), and/or of the compound 1,1,2,2-tetrafluoro-1-(trifluoromethoxy)ethane (TFTFME) (E 227), i.e., the precursor or intermediate compound of perfluoro(methyl vinyl ether) (PFMVE), respectively, as each defined herein and in the claims, wherein the reaction is carried out in at least one of the steps (A) and (B) as a continuous processes, wherein the continuous process is performed in at least one continuous flow reactor with upper lateral dimensions of about ≤5 mm, or of about ≤4 mm,

preferably in at least one microreactor;

more preferably wherein of the said steps at least (A) the step of a fluorination reaction is a continuous process in at least one microreactor under one or more of the following conditions:
flow rate: of from about 10 ml/h up to about 400 l/h;
temperature: ranging of from about −20° C. or of from about −10° C. or of from about 0° C. or of from about 10° C., or of from about 20° C. or of from about 30° C., respectively, each ranging to up to about 150° C.;
pressure: of from about 1 bar (1 atm abs.) up to about 50 bar; preferably of from about 1 bar (1 atm abs.) up to about 20 bar, more preferably at about 1 bar (1 atm abs.) up to about 5 bar; most preferably at about 1 bar (1 atm abs.) up to about 4 bar; in an example the pressure is about 3 bar;
residence time: of from about 1 second, preferably from about 1 minute, up to about 60 minutes.

The invention also relates to a process, as described herein, optionally either operated in a batch manner or operated in a continuous manner, for the manufacture of PFMVE (perfluoro(methyl vinyl ether)) having the formula (I), or process for the manufacture of compound 1,1,2,2-tetrafluoro-1-(trifluoromethoxy)ethane (TFTFME) (E 227) having the formula (II), characterized in that in step (A) the in the first reactor the addition reaction is performed in an SiC-reactor.

The invention also relates to a process, as described herein, optionally either operated in a batch manner or operated in a continuous manner, for the manufacture of PFMVE (perfluoro(methyl vinyl ether)) having the formula (I), or a process for the manufacture compound 1,1,2,2-tetrafluoro-1-(trifluoromethoxy)ethane (TFTFME) (E 227) having the formula (II), characterized in that in step (B) in the second reactor the elimination reaction is performed in a nickel-reactor (Ni-reactor) or in a reactor with an inner surface with high nickel-content (Ni-content).

The boiling point of the compound perfluoro(methyl vinyl ether) (PFMVE) is −22° C. (at normal or ambient pressure), and thus, at room temperature the compound perfluoro-(methyl vinyl ether) (PFMVE) is gaseous. Accordingly, in an embodiment of the process of the invention the compound perfluoro(methyl vinyl ether) (PFMVE) is isolated in that there is a cooler used after the reaction, e.g., after the HF-elimination step (B) reactor, to cool down the entire reaction mixture to 0° C. (cooler not shown in the Figures), and further in that most of the HF formed, e.g., in the HF-elimination step (B) is purged over a cyclone into a scrubber, and the compound perfluoro(methylvinylether) (PFMVE) is collected in a cooling trap kept at a temperature of below the boiling point of PFMVE at given pressure, for example, at or below the boiling point of PFMVE which is about −22° C. For example, the cooling trap is kept at a temperature lower of about −20° C., preferably at a temperature of about −30° C.

### BRIEF DESCRIPTION OF THE DRAWINGS

FIG. 1 shows the manufacture of PFVME out of HFE 254 (1,1,2,2-tetrafluoro-1-(methoxy)ethane) and F2 fluorination gas, over E 227 (TFTFME) as an intermediate compound, and using a counter current reactor system.

FIG. 2 shows the manufacture of PFMVE by reaction of HFE 254 (1,1,2,2-tetrafluoro-1-(methoxy)ethane) with F2 fluorination gas, over E 227 (TFTFME) as an intermediate compound in a sequence of two microreactors.

### DETAILED DESCRIPTION OF THE INVENTION

As briefly described in the Summary of the Invention, and defined in the claims and further detailed by the following description and examples herein, the invention relates to a new industrial process for manufacturing of perfluoro(methyl vinyl ether) (PFMVE), and/or of 1,1,2,2-tetrafluoro-1-(trifluoromethoxy)ethane (TFTFME) (E 227), which is a suitable intermediate in the manufacture of perfluoro(methyl vinyl ether) (PFMVE), involving reactions in liquid phase and performing reactions in a counter-current reactor system, in particular in a loop reactor system, or in a counter-current (loop) system (“inverse gas scrubber system”), as well as in a tube reactor system, a continuous flow reactor system, in a coil reactor system or in a microreactor system, preferably performing reactions in a counter-current reactor system or in a microreactor, respectively, as each described here under and in the claims.

In one aspect, the invention pertains to a process for the manufacture of the compound PFMVE (perfluoro(methyl vinyl ether)) having the formula (I),

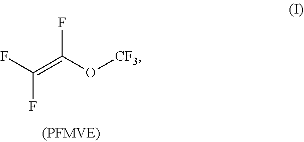

wherein the process comprises the steps of a direct fluorination reaction (A) and HF-elimination reaction (B), in a reactor or reactor system, resistant to elemental fluorine (F₂) and hydrogen fluoride (HF):

(A) in a first reaction step a direct fluorination by reacting the compound HFE-254 (1,1,2,2-tetrafluoro-1-(methoxy)ethane) of formula (III),

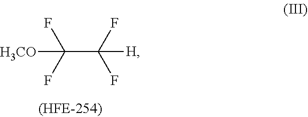

with an about stoichiometric quantity of elemental fluorine (F2) comprised in a fluorination gas to selectively substitute in the compound of formula (III) the three hydrogen atoms of the 1-(methoxy) group of the compound HFE-254 of formula (III) for fluorine, and wherein the reaction is carried out at temperature in the range of from about 0° C. to about +60° C.,

preferably at temperature in the range of from about 0° C. to about +60° C., more preferably of at temperature in the range from about 10° C. to about +50° C., even more preferably at temperature in the range of from about 20° C. to about +40° C.,

and at a pressure in the range of from about 1 bar absolute bar to about 20 bar absolute,

preferably at a pressure in the range of from about 5 bar absolute bar to about 20 bar absolute, more preferably at a pressure in the range of from about 5 bar absolute bar to about 15 bar absolute, even more preferably at a pressure in the range of from about 5 bar absolute bar to about 12 bar absolute, and most preferably at a pressure in the range of from about 6 bar absolute bar to about 11 bar absolute,

to yield the compound TFTFME (1,1,2,2-tetrafluoro-1-(trifluoromethoxy)ethane), (E 227), of formula (II),

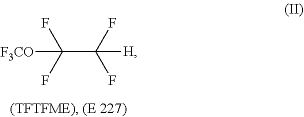

and

with or without isolating and/or purifying the (intermediate) fluorination product (TFTFME), (E 227); preferably without isolating and/or purifying the (intermediate) fluorination product (TFTFME), (E 227),

(B) in a second reaction step an elimination reaction, wherein HF (hydrogen fluoride) is eliminated from the (intermediate) fluorination product (TFTFME), (E 227), of formula (II), obtained in step (A), and the elimination reaction is performed

(i) as a(n) (exothermic) elimination reaction in the presence of one or more nitrogen-containing organic bases,

preferably as a non-aqueous (exothermic) elimination reaction in the presence of one or more nitrogen containing organic bases,

and/or

in the presence of one or more inorganic bases,

preferably as a(n) (exothermic) elimination reaction with an aqueous solution containing one or more inorganic bases, more preferably as a(n) (exothermic) elimination reaction with an aqueous solution containing one or more inorganic bases in the presence of one or more phase transfer catalysts,

wherein the temperature of the (exothermic) elimination reaction is controlled to not exceed a temperature of about 60° C.,

preferably to not exceed a temperature of about 50° C., more preferably to not exceed a temperature of about 45° C., and even more preferably to not exceed a temperature of about 40° C.,

and wherein the (exothermic) elimination reaction is carried out at a pressure in the range of from about 1 bar absolute bar to about 20 bar absolute,

preferably at a pressure in the range of from about 4 bar absolute bar to about 20 bar absolute, more preferably at a pressure in the range of from about 4 bar absolute bar to about 15 bar absolute, even more preferably at a pressure in the range of from about 4 bar absolute bar to about 10 bar absolute, and most preferably at a pressure in the range of from about 4 bar absolute bar to about 8 bar absolute,

or

(ii) as a, non-catalytic or preferably catalytic, more preferably Ni (nickel) catalytic, thermal elimination reaction at a temperature in the range of about 60° C. to about 120° C.,

preferably at a temperature in the range of about 70° C. to about 110° C., more preferably at a temperature in the range of about 70° C. to about 100° C., even more preferably at a temperature in the range of about 70° C. to about 90° C.,

to yield the compound PFMVE (perfluoro(methyl vinyl ether)) of formula (I),

and

(C) withdrawing and collecting the compound PFMVE (perfluoro(methyl vinyl ether)) of formula (I) obtained in step (B) from the reactor or reactor system,

and

(D) optionally isolating and/or purifying the compound PFMVE (perfluoro(methyl vinyl ether)) of formula (I).

In another aspect, the invention pertains to a process for the manufacture of the compound PFMVE (perfluoro(methyl vinyl ether)) having the formula (I),

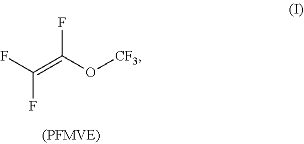

wherein the process comprises, in a reactor or reactor system, resistant to elemental fluorine (F₂) and hydrogen fluoride (HF), performing an HF-elimination reaction step (B), wherein in the elimination reaction, HF (hydrogen fluoride) is eliminated from the compound TFTFME (1,1,2,2-tetrafluoro-1-(trifluoromethoxy)ethane), (E 227), of formula (II),

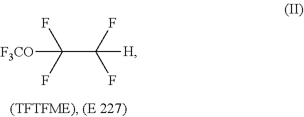

and the elimination reaction step (B) is performed

(i) as a(n) (exothermic) elimination reaction in the presence of one or more nitrogen-containing organic bases,

preferably as a non-aqueous (exothermic) elimination reaction in the presence of one or more nitrogen containing organic bases,

and/or

in the presence of one or more inorganic bases,

preferably as a(n) (exothermic) elimination reaction with an aqueous solution containing one or more inorganic bases, more preferably as a(n) (exothermic) elimination reaction with an aqueous solution containing one or more inorganic bases in the presence of one or more phase transfer catalysts,

wherein the temperature of the (exothermic) elimination reaction is controlled to not exceed a temperature of about 60° C.,

preferably to not exceed a temperature of about 50° C., more preferably to not exceed a temperature of about 45° C., and even more preferably to not exceed a temperature of about 40° C.,

and wherein the (exothermic) elimination reaction is carried out at a pressure in the range of from about 1 bar absolute bar to about 20 bar absolute,

preferably at a pressure in the range of from about 4 bar absolute bar to about 20 bar absolute, more preferably at a pressure in the range of from about 4 bar absolute bar to about 15 bar absolute, even more preferably at a pressure in the range of from about 4 bar absolute bar to about 10 bar absolute, and most preferably at a pressure in the range of from about 4 bar absolute bar to about 8 bar absolute,

or

(ii) as a, non-catalytic or preferably catalytic, more preferably Ni (nickel) catalytic, thermal elimination reaction at a temperature in the range of about 60° C. to about 120° C.,

preferably at a temperature in the range of about 70° C. to about 110° C., more preferably at a temperature in the range of about 70° C. to about 100° C., even more preferably at a temperature in the range of about 70° C. to about 90° C.,

to yield the compound PFMVE (perfluoro(methyl vinyl ether)) of formula (I),

and

(C) withdrawing and collecting the compound PFMVE (perfluoro(methyl vinyl ether)) of formula (I) obtained in step (B) from the reactor or reactor system, and

(D) optionally isolating and/or purifying the compound PFMVE (perfluoro(methyl vinyl ether)) of formula (I).

In a further aspect, the invention pertains to a process for the manufacture of the compound TFTFME (1,1,2,2-tetrafluoro-1-(trifluoromethoxy)ethane), (E 227), of formula (II),

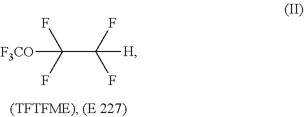

wherein the process comprises, in a reactor or reactor system, resistant to elemental fluorine (F₂) and hydrogen fluoride (HF), performing a direct fluorination reaction step (A), wherein in the direct fluorination reaction the compound HFE-254 (1,1,2,2-tetrafluoro-1-(methoxy)ethane) of formula (III),

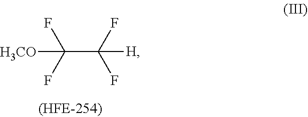

is fluorinated with an about stoichiometric quantity of elemental fluorine (F₂) comprised in a fluorination gas to selectively substitute in the compound of formula (III) the three hydrogen atoms of the 1-(methoxy) group of the compound HFE-254 of formula (III) for fluorine, and wherein the reaction is carried out at temperature in the range of from about 0° C. to about +60° C.,

preferably at temperature in the range of from about 0° C. to about +60° C., more preferably of at temperature in the range from about 10° C. to about +50° C., even more preferably at temperature in the range of from about 20° C. to about +40° C.,

and at a pressure in the range of from about 1 bar absolute bar to about 20 bar absolute,

preferably at a pressure in the range of from about 5 bar absolute bar to about 20 bar absolute, more preferably at a pressure in the range of from about 5 bar absolute bar to about 15 bar absolute, even more preferably at a pressure in the range of from about 5 bar absolute bar to about 12 bar absolute, and most preferably at a pressure in the range of from about 6 bar absolute bar to about 11 bar absolute,

to yield the compound TFTFME (1,1,2,2-tetrafluoro-1-(trifluoromethoxy)ethane), (E 227), of formula (II),

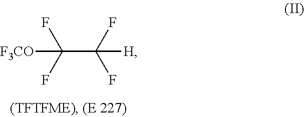

and

(C) withdrawing and collecting the compound TFTFME (1,1,2,2-tetrafluoro-1-(trifluoromethoxy)ethane), (E 227), of formula (II) obtained in step (A) from the reactor or reactor system,

and

(D) optionally isolating and/or purifying the compound TFTFME (1,1,2,2-tetrafluoro-1-(trifluoromethoxy)ethane), (E 227), of formula (II).

In another aspect, the invention also pertains to a process as defined here before, for the manufacture of the compound PFMVE (perfluoro(methyl vinyl ether)) having the formula (I), or for the manufacture of the compound TFTFME (1,1,2,2-tetrafluoro-1-(trifluoromethoxy)ethane), (E 227), of formula (II), wherein the direct fluorination reaction (A) and/or the HF-elimination reaction (B) is carried out in a (closed) column reactor.

In yet another aspect, the invention pertains to a process as defined here before, for the manufacture of the compound PFMVE (perfluoro(methyl vinyl ether)) having the formula (I), or the process according to claim 3 for the manufacture of the compound TFTFME (1,1,2,2-tetrafluoro-1-(trifluoromethoxy)ethane), (E 227), of formula (II), wherein the liquid reaction medium of the direct fluorination reaction (A) is circulated in a loop in a (closed) column reactor to perform the fluorination reaction (A), while the fluorination gas comprising elemental fluorine (F₂) is fed into the (closed) column reactor and is passed through the liquid reaction medium to react with the compound HFE-254 (1,1,2,2-tetrafluoro-1-(methoxy)ethane) of formula (III); preferably wherein the loop is operated with a circulation velocity in the range of from about 1,000 l/h to about 2,000 l/h, more preferably in the range of from about 1,250 l/h to about 1,750 l/h; still more preferably wherein the loop is operated with a circulation velocity in the range of from about 1,500 l/h+200 l/h; even more preferably wherein the loop is operated with a circulation velocity in the range of from about 1,500 l/h+100 l/h; and most preferably wherein the loop is operated with a circulation velocity in the range of from about 1,500 l/h±50 l/h.

For example, in said yet another aspect of the invention as defined here before, pertains to a process, wherein for the direct fluorination reaction (A) the (closed) column reactor is equipped with at least one of the following:

(i) at least one heat exchanger (system), at least one liquid reservoir, with inlet and outlet for, and containing the liquid reaction medium,

e.g., initially comprising or consisting of the compound HFE-254 (1,1,2,2-tetrafluoro-1-(methoxy)ethane) of formula (III) or as the reaction proceeds increasingly comprising or consisting of the compound TFTFME (1,1,2,2-tetrafluoro-1-(trifluoromethoxy)ethane), (E 227), of formula (II);

(ii) a pump for pumping and circulating the liquid reaction medium;

(iii) one or more (nozzle) jets, preferably wherein the one or more (nozzle) jets are placed at the top of the column reactor, for spraying the circulating reaction medium into the (closed) column reactor;

(iv) one or more feeding inlets for introducing the fluorination gas comprising or consisting of elemental fluorine (F2) into the (closed) column reactor;

(v) optionally one or more sieves, preferably two sieves, preferably the one or more sieves placed at the bottom of the (closed) column reactor;

(vi) and at least one gas outlet equipped with a pressure valve, and at least one outlet for withdrawing the fluorinated compound TFTFME (1,1,2,2-tetrafluoro-1-(trifluoromethoxy)ethane), (E 227), of formula (II) from the (closed) column reactor.

As already described above, when performing fluorination (A) reactions in a counter-current reactor system, in particular in a loop reactor system, or in a counter-current (loop) system (“inverse gas scrubber system”), the fluorination (A) reactions can be performed over the whole wide range of fluorine (F₂) concentration in the F₂-fluorination gas of from about 1% by volume of elemental fluorine (F₂) up to about almost 100% by volume of elemental fluorine (F₂), based on the total F₂-fluorination gas composition as 100% by volume.

In this aspect, for example, the invention pertains to a process for the manufacture of the compound PFMVE (perfluoro(methyl vinyl ether)) having the formula (I), or a process for the manufacture of the compound TFTFME (1,1,2,2-tetrafluoro-1-(trifluoromethoxy)-ethane), (E 227), of formula (II), wherein the fluorination (A) reaction is performed in a counter-current reactor system, in particular in a loop reactor system, or in a counter-current (loop) system (“inverse gas scrubber system”), and wherein the fluorine (F₂) concentration in the F₂-fluorination gas is in a range of from about 1% by volume of elemental fluorine (F₂) up to about almost 100% by volume of elemental fluorine (F₂), based on the total F₂-fluorination gas composition as 100% by volume;

preferably wherein

(i) the fluorine (F2) concentration in the F2-fluorination gas is in a range of from about 1% by volume of elemental fluorine (F2) up to about 30% by volume of elemental fluorine (F2), more preferably of from about 5% by volume of elemental fluorine (F2) up to about 25% by volume of elemental fluorine (F2), even more preferably of from about 5% by volume of elemental fluorine (F2) up to about 20% by volume of elemental fluorine (F2), each range based on the total F2-fluorination gas composition as 100% by volume; or

(ii) the fluorine (F2) concentration in the F2-fluorination gas is in a range of from about 85% by volume of elemental fluorine (F2) up to about almost 100% by volume of elemental fluorine (F2), more preferably of from about 90% by volume of elemental fluorine (F2) up to about almost 100% by volume of elemental fluorine (F2), based on the total F2-fluorination gas composition as 100% by volume.

Accordingly, when performing fluorination (A) reactions in a counter-current reactor system, in particular in a loop reactor system, or in a counter-current (loop) system (“inverse gas scrubber system”), in one aspect the invention also pertains to a process as defined above, for the manufacture of the compound PFMVE (perfluoro(methyl vinyl ether)) having the formula (I), or for the manufacture of the compound TFTFME (1,1,2,2-tetrafluoro-1-(trifluoromethoxy)ethane), (E 227), of formula (II), wherein the lower fluorine (F₂) concentration in the F₂-fluorination gas is applied, and wherein the fluorination gas in the direct fluorination reaction step (A) is elemental fluorine (F₂) diluted in one or more inert gases, and wherein the elemental fluorine (F₂) is present in the fluorination gas in a concentration in a range of from about 1% up to about 30% by volume of elemental fluorine (F₂), preferably of from about 5% up to about 25% by volume of elemental fluorine (F₂), more preferably of from about 5% up to about 20% by volume of elemental fluorine (F₂), each range based on the total F₂-fluorination gas composition as 100% by volume. Even more preferably, when performing reactions in said counter-current reactor system, in particular loop reactor system, or counter-current (loop) system (“inverse gas scrubber system”), the fluorination gas in the direct fluorination reaction step (A) is elemental fluorine (F₂) diluted in one or more inert gases, and the elemental fluorine (F₂) is present in the fluorination gas in a concentration in a range of from about 5% up to about 15% by volume of elemental fluorine (F₂), still more preferably in a range of about 8% up to about 15% by volume of elemental fluorine (F₂), and most preferably in a range of about 8% up to about 12% by volume of elemental fluorine (F₂), e.g., the elemental fluorine (F₂) is present in the fluorination gas in a concentration of about 10% by volume (e.g., 10±2% by volume, or 10±1% by volume, respectively). It goes without saying that a skilled person will understand that within any of the above given ranges any intermediate values and intermediate ranges can be selected, too.

Accordingly, when performing fluorination (A) reactions in a counter-current reactor system, in particular in a loop reactor system, or in a counter-current (loop) system (“inverse gas scrubber system”), in another aspect, the invention also pertains to a process as defined above, for the manufacture of the compound PFMVE (perfluoro(methyl vinyl ether)) having the formula (I), or for the manufacture of the compound TFTFME (1,1,2,2-tetrafluoro-1-(trifluoromethoxy)ethane), (E 227), of formula (II), wherein the higher fluorine (F₂) concentration in the F₂-fluorination gas is applied, and wherein the elemental fluorine (F₂) is present in the fluorination gas in a concentration in a range of from about 85% up to about almost 100% (as defined herein above) by volume of elemental fluorine (F₂), most preferably of from about 90% by volume of elemental fluorine (F₂) up to about almost 100% (as defined herein above) by volume of elemental fluorine (F₂), based on the total F₂-fluorination gas composition as 100% by volume. Even more preferably, when performing reactions in said counter-current reactor system, in particular loop reactor system, or counter-current (loop) system (“inverse gas scrubber system”), the F₂-fluorination gas used in the fluorination process step (A) of the invention, for example, is a fluorine (F₂) gas only to some extent diluted in an inert gas (together then they constitute the F₂-fluorination gas), with fluorine (F₂) concentrations in ranges, for example, with a maximum concentration of up to about almost 100% by volume of elemental fluorine (F₂), in the range of from about 85% by volume, in particular in the range of from about 90% by volume or in particular in the range of from about 92% by volume of elemental fluorine (F₂), especially in the range of from about 94% by volume; each given range based on the fluorine (F₂) gas and the inert gas as 100% by volume, i.e., based on the total F₂-fluorination gas composition as 100% by volume.

In another aspect, when performing fluorination (A) reactions in a counter-current reactor system, in particular in a loop reactor system, or in a counter-current (loop) system (“inverse gas scrubber system”), the invention also pertains to a process as defined above, for the manufacture of the compound PFMVE (perfluoro(methyl vinyl ether)) having the formula (I), or for the manufacture of the compound TFTFME (1,1,2,2-tetrafluoro-1-(trifluoromethoxy)ethane), (E 227), of formula (II), wherein the higher fluorine (F₂) concentration in the F₂-fluorination gas is applied, and wherein, in a very practical range, for example, in particular if the F₂-fluorination gas is derived from an F₂-electrolysis reactor (fluorine cell), purified or unpurified, and wherein the fluorine (F₂) gas from the F₂-electrolysis reactor (fluorine cell) is only to some extent diluted in an inert gas (together then they constitute the F₂-fluorination gas), with fluorine (F₂) in a concentration within a range of from about 92% by volume of elemental fluorine (F₂) up to about 99% by volume of elemental fluorine (F₂), and most preferably in a very practical range of from about 94% by volume to about 99% by volume; each given range based on the fluorine (F₂) gas and the inert gas as 100% by volume, i.e., based on the total F₂-fluorination gas composition as 100% by volume.

It goes without saying that a skilled person will understand that within any of the above given ranges any intermediate values and intermediate ranges can be selected, too.

In still another aspect, the invention pertains to a process as defined here before, for the manufacture of the compound PFMVE (perfluoro(methyl vinyl ether)) having the formula (I), wherein the liquid reaction medium of the HF-elimination reaction (B) is circulated in a loop in a (closed) column reactor to perform the HF-elimination reaction (B), and wherein the loop is operated with a circulation velocity in the range of from about 1,000 l/h to about 2,000 l/h, preferably in the range of from about 1,250 l/h to about 1,750 l/h; more preferably wherein the loop is operated with a circulation velocity in the range of from about 1,500 l/h+200 l/h; even more preferably wherein the loop is operated with a circulation velocity in the range of from about 1,500 l/h+100 l/h; and most preferably wherein the loop is operated with a circulation velocity in the range of from about 1,500 l/h±50 l/h.

For example, in said still another aspect of the invention as defined here before, pertains to a process, wherein for the HF-elimination reaction (B) the (closed) column reactor is equipped with at least one of the following:

(i) at least one heat exchanger (system), at least one liquid reservoir, with inlet and outlet for, and containing the liquid reaction medium,

e.g., initially comprising or consisting of the compound TFTFME (1,1,2,2-tetrafluoro-1-(trifluoromethoxy)ethane), (E 227), of formula (II), or as the reaction proceeds increasingly comprising or consisting of the compound PFMVE (perfluoro(methyl vinyl ether)) having the formula (I);

(ii) a pump for pumping and circulating the liquid reaction medium;

(iii) one or more (nozzle) jets, preferably wherein the one or more (nozzle) jets are placed at the top of the column reactor, for spraying the circulating reaction medium into the (closed) column reactor;

(iv) optionally, in case of (i) preferably performing the HF-elimination reaction as a(n) (exothermic) elimination reaction in the presence of one or more nitrogen-containing organic bases, one or more feeding inlets for introducing the one or more nitrogen-containing organic bases into the (closed) column reactor;

(v) optionally one or more sieves, preferably two sieves, preferably the one or more sieves placed at the bottom of the (closed) column reactor;

(vi) and at least one gas outlet equipped with a pressure valve, and at least one outlet for withdrawing the compound PFMVE (perfluoro(methyl vinyl ether)) having the formula (I) from the (closed) column reactor.

In an aspect of the invention, wherein the direct fluorination reaction (A) and/or the HF-elimination reaction (B) is carried out in a (closed) column reactor, the invention pertains to a process as defined here before, for the manufacture of the compound PFMVE (perfluoro(methyl vinyl ether)) having the formula (I), or for the manufacture of the compound TFTFME (1,1,2,2-tetrafluoro-1-(trifluoromethoxy)ethane), (E 227), of formula (II), wherein column reactor is a packed bed tower reactor, preferably a packed bed tower reactor is packed with fillers resistant to the reactants and especially resistant to elemental fluorine (F₂) and to hydrogen fluoride (HF) such as, e.g., with Raschig fillers, E-TFE fillers, and/or HF-resistant metal fillers, e.g., Hastelloy metal fillers, and/or (preferably) HDPTFE-fillers, more preferably wherein the packed bed tower reactor is a gas scrubber system (tower) which is packed with any of the before mentioned HF-resistant Hastelloy metal fillers and/or HDPTFE-fillers, and preferably with HDPTFE-fillers.

In yet a further aspect the invention pertains to a process as defined here before, for the manufacture of the compound PFMVE (perfluoro(methyl vinyl ether)) having the formula (I), or for the manufacture of the compound TFTFME (1,1,2,2-tetrafluoro-1-(trifluoromethoxy)ethane), (E 227), of formula (II), wherein the direct fluorination reaction (A) and/or the HF-elimination reaction (B) is carried out in at least one step in a continuous flow reactor with upper lateral dimensions of about ≤5 mm, or of about ≤4 mm, more preferably in at least one step in a microreactor;

still more preferably wherein the direct fluorination reaction (A) and/or the HF-elimination reaction (B) is carried out in at least in one step as a continuous processes, wherein the continuous process is performed in at least one continuous flow reactor with upper lateral dimensions of about ≤5 mm, or of about ≤4 mm;

even more preferably wherein the direct fluorination reaction (A) and/or the HF-elimination reaction (B) is carried out in at least in one step as a continuous processes, wherein the continuous process is performed in at least one microreactor.

In still a further aspect the invention pertains to a process as defined here before, for the manufacture of the compound PFMVE (perfluoro(methyl vinyl ether)) having the formula (I), or for the manufacture of the compound TFTFME (1,1,2,2-tetrafluoro-1-(trifluoromethoxy)ethane), (E 227), of formula (II), characterized in that prior to starting any of the process steps (A) and (B) one or more of the reactors used, preferably each and any of the reactors used, are purged with an inert gas or a mixture of inert gases, preferably with He (helium) and/or N₂ (nitrogen) as the inert gas, more preferably with N₂ (nitrogen) as the inert gas.

In a particular aspect the invention pertains to a process as defined here before, for the manufacture of the compound PFMVE (perfluoro(methyl vinyl ether)) having the formula (I), or for the manufacture of the compound TFTFME (1,1,2,2-tetrafluoro-1-(trifluoromethoxy)ethane), (E 227), of formula (II), characterized in that in the fluorination reaction step (A) the reaction is performed in a SiC-reactor; preferably in that in the fluorination reaction step (A) the reaction is performed in a SiC-microreactor.

In another particular aspect the invention pertains to a process as defined here before, for the manufacture of the compound PFMVE (perfluoro(methyl vinyl ether)) having the formula (I), characterized in that in the HF-elimination step (B) the reaction is performed in a nickel-reactor (Ni-reactor) or in a reactor with an inner surface with high nickel-content (Ni-content); preferably in that in the HF-elimination step (B) the reaction is performed in a nickel-microreactor (Ni-microreactor) or in a microreactor with an inner surface with high nickel-content (Ni-content).

In a further particular aspect the invention pertains to a process as defined here before, for the manufacture of the compound PFMVE (perfluoro(methyl vinyl ether)) having the formula (I), or for the manufacture of the compound TFTFME (1,1,2,2-tetrafluoro-1-(trifluoromethoxy)ethane), (E 227), of formula (II), characterized in that, independently, the product yielding from fluorination reaction step (A) and/or the product yielding from HF-elimination step (B) are subjected to distillation.

In still another aspect, the invention pertains also to any one of the above defined processes for the manufacture of PFMVE (perfluoro(methyl vinyl ether)) having the formula (I), or also to any one of the above defined processes for the manufacture of 1,1,2,2-tetrafluoro-1-(trifluoromethoxy)ethane (TFTFME) (E 227) having the formula (II), characterized in that in step (B) in the second reactor the elimination reaction is performed in a nickel-reactor (Ni-reactor) or in a reactor with an inner surface with high nickel-content (Ni-content). Preferably, in the context of the present invention the term “high nickel-content” means a nickel (Ni) content of at least 50% in the metal alloy the nickel-reactor is made of Particularly preferred is a nickel-reactor made out of Hastelloy C4 nickel alloy. The Hastelloy C4 nickel alloy is known in the state of the art to be a nickel alloy comprising a combination of chromium with high molybdenum content. Such Hastelloy C4 nickel alloy shows exceptional resistance to a large number of chemical media such as contaminated, reducing mineral acids, chlorides and organic and inorganic media contaminated with chloride.

Hastelloy C4 nickel alloy is commercially available, for example, under the tradenames Nicrofer® 6616 hMo or Hastelloy C-4®, respectively. The density of Hastelloy C4 nickel alloy is 8.6 g/cm³, and the melting temperature range is 1335 to 1380° C.

Due to its special chemical composition of C4, the Hastelloy C4 nickel alloy has good structural stability and high resistance to sensitization.

The chemical composition of Hastelloy C4 (nickel alloy), for example, is in the following Table 1, wherein the nickel (Ni) content is at least 50% in the metal alloy, and the nickel (Ni) content is adding up the Hastelloy C4 nickel alloy compositions to a total of 100% metal alloy.

TABLE 1
Chemical composition of Hastelloy C4 (nickel alloy).

| C | Si | Mn | P | S | Cr | Mo | Co | Fe | Ti |
|---|---|---|---|---|---|---|---|---|---|
| % | ≤% | ≤% | ≤% | ≤% | % | % | ≤% | % | % |
| 0-0.009 | 0-0.05 | 0-1.0 | 0-0.02 | 0-0.01 | 14.5-17.5 | 14.0-17.0 | 0-2.0 | 0-3 | 0-0.7 |

and nickel (Ni) as the remainder for adding up to 100% metal alloy.

In a further aspect, the invention pertains also to any one of the above defined processes for the manufacture of PFMVE (perfluoro(methyl vinyl ether)) having the formula (I), or also to any one of the above defined processes for the manufacture of 1,1,2,2-tetrafluoro-1-(trifluoromethoxy)ethane (TFTFME) (E 227) having the formula (II), characterized in that in step (A) the fluorination reaction is performed in a continuous manner, preferably in a continuous manner in a microreactor.

In another particular and preferred aspect, the invention pertains also to any one of the above defined processes for the manufacture of PFMVE (perfluoro(methyl vinyl ether)) having the formula (I), characterized in that in step (B) the elimination reaction is performed in a continuous manner, preferably in a continuous manner in a microreactor.

In yet another particular and preferred aspect, the invention pertains also to any one of the above defined processes for the manufacture of PFMVE (perfluoro(methyl vinyl ether)) having the formula (I), or also to any one of the above defined processes for the manufacture of 1,1,2,2-tetrafluoro-1-(trifluoromethoxy)ethane (TFTFME) (E 227) having the formula (II), characterized in that, independently the reaction in at least one reaction step of (A) and (B) is carried as a continuous processes, wherein the continuous process in the at least one reaction step of (A) and (B) is performed in at least one continuous flow reactor with upper lateral dimensions of about ≤5 mm, or of about ≤4 mm, preferably wherein at least one of the continuous flow reactor is a microreactor.

In a more preferred aspect, the invention pertains also to any one of the above defined processes for the manufacture of PFMVE (perfluoro(methyl vinyl ether)) having the formula (I), or also to any one of the above defined processes for the manufacture of 1,1,2,2-tetrafluoro-1-(trifluoromethoxy)ethane (TFTFME) (E 227) having the formula (II), characterized in that the reaction is carried out in at least one reaction step of (A) and (B) as a continuous processes, wherein the continuous process is performed in at least one continuous flow reactor with upper lateral dimensions of about ≤5 mm, or of about ≤4 mm, preferably in at least one microreactor;

more preferably wherein of the said steps of (A) and (B) at least the step (A) of a fluorination reaction is a continuous process in at least one microreactor under one or more of the following conditions:
flow rate: of from about 10 ml/h up to about 400 l/h;
temperature: ranging of from about −20° C. or of from about −10° C. or of from about 0° C. or of from about 10° C., or of from about 20° C. or of from about 30° C., respectively, each ranging to up to about 150° C.;
pressure: of from about 1 bar (1 atm abs.) up to about 50 bar; preferably of from about 1 bar (1 atm abs.) up to about 20 bar, more preferably at about 1 bar (1 atm abs.) up to about 5 bar; most preferably at about 1 bar (1 atm abs.) up to about 4 bar; in an example the pressure is about 3 bar;
residence time: of from about 1 second, preferably from about 1 minute, up to about 60 minutes.

In a further aspect, the invention pertains also to any one of the above defined processes for the manufacture of PFMVE (perfluoro(methyl vinyl ether)) having the formula (I), or also to any one of the above defined processes for the manufacture of 1,1,2,2-tetrafluoro-1-(trifluoromethoxy)ethane (TFTFME) (E 227) having the formula (II), characterized in that, independently, the product yielding from step (A) and/or the product yielding from step (B) are subjected to distillation.

Batch Process:

The invention also may pertain to a process for the manufacture of a fluorinated compound, comprising a particular process step which is performed batchwise, preferably wherein the batchwise process step is carried out in a column reactor. Although, in the following column reactor setting the process is described as a batch process, optionally the process can be performed in the said column reactor setting also as a continuous process. In case of a continuous process in the said column reactor setting, then, it goes without saying, the additional inlet(s) and outlet(s) are foreseen, for feeding the starting compound and withdrawing the product compound, respectively, and/or if desired any intermediate compound. Reference is made to Example 9.

If the invention pertains to a batchwise process, preferably wherein the batchwise process is carried out in a column reactor, the process for manufacturing of perfluoro(methylvinylether) (PFMVE), and/or of 1,1,2,2-tetrafluoro-1-(trifluoromethoxy)ethane (TFTFME) (E 227), which is a suitable intermediate in the manufacture of perfluoro(methylvinylether) (PFMVE), most preferably the reaction is carried out in a (closed) column reactor (system), wherein the liquid medium comprising or consisting of a liquid starting compound, e.g., the compound HFE-254 (1,1,2,2-tetrafluoro-1-(methoxy)ethane) or TFTFME (E 227), respectively, as a liquid medium is circulated in a loop; preferably wherein the loop in the column reactor is operated with a circulation velocity of from 1,500 l/h to 5,000 l/h, more preferably of from 3,500 l/h to 4,500 l/h.

If the invention pertains to such a batchwise process, the process for manufacturing of perfluoro(methyl vinyl ether) (PFMVE) and/or of 1,1,2,2-tetrafluoro-1-(trifluoromethoxy)ethane (TFTFME) (E 227) according to the invention can be carried out such that the mentioned liquid medium is circulated in the column reactor in a turbulent stream or in laminar stream, preferably in a turbulent stream.

In general, a gaseous starting compound, e.g., the F₂-fluorination gas, respectively, is fed into the loop in accordance with the required stoichiometry for the targeted product compound and/or if desired any intermediate compound, and adapted to the reaction rate.

For example, the said process for the manufacture of a compound PFMVE and/or TFTFME (E 227) according to the invention, may be performed, e.g., batchwise, wherein the column reactor is equipped with at least one of the following: at least one cooler (system), at least one liquid reservoir for the liquid medium comprising or consisting of a liquid starting compound, a pump (for pumping/circulating the liquid medium), one or more (nozzle) jets, preferably placed at the top of the column reactor, for spraying the circulating medium into the column reactor, one or more feeding inlets for introducing a gaseous starting compound, e.g., F₂-fluorination gas, optionally one or more sieves, preferably two sieves, preferably the one or more sieves placed at the bottom of the column reactor, and at least one gas outlet equipped with a pressure valve.

Accordingly, the process for manufacturing of perfluoro(methyl vinyl ether) (PFMVE) and/or of 1,1,2,2-tetrafluoro-1-(trifluoromethoxy)ethane (TFTFME) (E 227) compound according to the invention, can be performed in column reactor which is equipped with at least one of the following:

(i) at least one cooler (system), at least one liquid reservoir, with inlet and outlet for, and containing the liquid medium comprising or consisting of a starting compound; preferably the compound HFE-254 (1,1,2,2-tetrafluoro-1-(methoxy)ethane) or TFTFME (E 227), respectively;

(ii) a pump for pumping and circulating the liquid medium in the column reactor;

(iii) one or more (nozzle) jets, preferably wherein the one or more (nozzle) jets are placed at the top of the column reactor, for spraying the circulating liquid medium into the column reactor;

(iv) one or more feeding inlets for introducing a gaseous compound, e.g., inert gas or a F2-fluorination gas, respectively into the column reactor;

(v) optionally one or more sieves, preferably two sieves, preferably the one or more sieves placed at the bottom of the column reactor;

(vi) and at least one gas outlet equipped with a pressure valve, and at least one outlet for withdrawing the product compound, respectively, and/or if desired any intermediate compound.

In one embodiment, the process for manufacturing of perfluoro(methyl vinyl ether) (PFMVE) and/or of 1,1,2,2-tetrafluoro-1-(trifluoromethoxy)ethane (TFTFME) (E 227) compound according to the invention can be performed in a column reactor which is a packed bed tower reactor, preferably a packed bed tower reactor which is packed with fillers (the terms “filler” and “filling”, are meant synonymously in the context of the invention) resistant to the reactants and especially resistant to hydrogen fluoride (HF). Fillers resistant to the reactants and especially resistant to hydrogen fluoride (HF) suitable in the context of the present invention are in particular HF-resistant plastic fillers and/or HF-resistant metal fillers. For example, under certain circumstances the packed bed tower reactor may be packed with stainless steel (1.4571) fillers, but stainless steel (1.4571) fillers are less suitable than other fillers mentioned herein after, because of possible risk of (minor) traces of humidity in the reactor system. Preferably, for example, in the invention the packed bed tower reactor is packed with fillers resistant to the reactants and especially resistant to hydrogen fluoride (HF) such as, e.g., with Raschig fillers, E-TFE fillers, and/or HF-resistant metal fillers, e.g., Hastelloy metal fillers, and/or (preferably) HDPTFE-fillers, more preferably wherein the packed bed tower reactor is a gas scrubber system (tower) which is packed with any of the before mentioned HF-resistant Hastelloy metal fillers and/or HDPTFE-fillers, and preferably with HDPTFE-fillers.

In a further embodiment, the process for manufacturing of perfluoro(methyl vinyl ether) (PFMVE) and/or of 1,1,2,2-tetrafluoro-1-(trifluoromethoxy)ethane (TFTFME) (E 227) compound according to the invention, the reaction is carried out with a counter-current flow of the circulating liquid medium comprising or consisting of the liquid starting compound and of the F₂-fluorination gas, respectively, that are fed into the column reactor.

The pressure valve functions to keep the pressure, as required in the reaction, and to release any effluent gas, e.g. inert carrier gas contained in the fluorination gas, if applicable together with any hydrogen halogenide gas released from the reaction.

The said process for manufacturing of perfluoro(methyl vinyl ether) (PFMVE) and/or of 1,1,2,2-tetrafluoro-1-(trifluoromethoxy)ethane (TFTFME) (E 227) compound according to the invention, may be performed, e.g., batchwise, such that in the said process the column reactor is a packed bed tower reactor as mentioned before, preferably a packed bed tower reactor which is packed with HDPTFE-fillers.

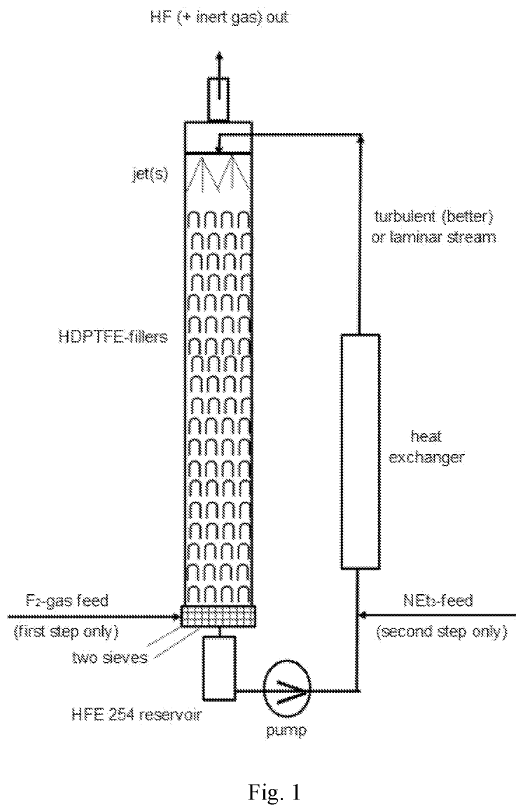

The packed tower according to FIG. 1 can have a diameter of 100 or 200 mm (depending on the circulating flow rate and scale) made out of Hastelloy C4 (nickel alloy)(known to the person skilled in the art), and has a length of 3 meters for the 100 mm and a length of 6 meters for the 200 mm diameter tower (latter if higher capacities are needed). The tower made out of Hastelloy is filled either with any of the fillings as mentioned before, or with the preferred HDPTFE-fillers, each of 10 mm diameter as commercially available. The size of fillings is quite flexible. The type of fillings is also quite flexible, within the boundaries of properties as stated herein above, i.e., the HDPTFE-fillers (or HDPTFE-fillings, respectively) were used in the trials disclosed hereunder in Example 1, and showed same performance, not causing much pressure reduction (pressure loss) while feeding any gaseous (starting) compound in counter-current manner.

Methods in a Continuous Flow Reactor System, e.g., Microreactor System:

The methods of the present invention as preferably described with microreactor are applicable to a continuous flow reactor system, as well as to a tube reactor system, and also applicable also to variants with coiled reactor system.

As already described above, when performing fluorination (A) reactions in a tube reactor system, in a continuous flow reactor system, in a coil reactor system, or in a microreactor system, preferably in a microreactor system, preferably the higher fluorine (F₂) concentration in the F₂-fluorination gas (as defined above) is adjusted when performing the fluorination (A) reactions.

In this aspect, for example, the invention pertains to a process for the manufacture of the compound PFMVE (perfluoro(methyl vinyl ether)) having the formula (I), or the process for the manufacture of the compound TFTFME (1,1,2,2-tetrafluoro-1-(trifluoromethoxy)ethane), (E 227), of formula (II), wherein the fluorination (A) reaction is performed in a tube reactor system, in a continuous flow reactor system, in a coil reactor system, or in a microreactor system, preferably in a microreactor system, and wherein the fluorine (F₂) concentration in the F₂-fluorination gas is in a range of from about 85% by volume of elemental fluorine (F₂) up to about almost 100% by volume of elemental fluorine (F₂), more preferably of from about 90% by volume of elemental fluorine (F₂) up to about almost 100% by volume of elemental fluorine (F₂), based on the total F₂-fluorination gas composition as 100% by volume.

Accordingly, when performing fluorination (A) reactions in a tube reactor system, in a continuous flow reactor system, in a coil reactor system, or in a microreactor system, preferably in a microreactor system, the F₂-fluorination gas used in the fluorination process step (A) of the invention, for example, is a fluorine (F₂) gas only to some extent diluted in an inert gas (together then they constitute the F₂-fluorination gas), with fluorine (F₂) concentrations in ranges, for example, with a maximum concentration of up to about almost 100% by volume of elemental fluorine (F₂), in the range of from about 85% by volume, in particular in the range of from about 90% by volume or in particular in the range of from about 92% by volume of elemental fluorine (F₂), especially in the range of from about 94% by volume; each given range based on the fluorine (F₂) gas and the inert gas as 100% by volume, i.e., based on the total F₂-fluorination gas composition as 100% by volume.

Accordingly, when performing fluorination (A) reactions in a tube reactor system, in a continuous flow reactor system, in a coil reactor system, or in a microreactor system, preferably in a microreactor system, the said fluorination process step (A) of the invention, for example, a fluorine (F₂) gas is only to some extent diluted in an inert gas (together then they constitute the F₂-fluorination gas), with fluorine (F₂) in a concentration more preferably within a range of from about 92% by volume to about almost 100% by volume, even more preferably within a range of from about 94% by volume to about almost 100% by volume, still more preferably in a very practical range, for example, in particular if the F₂-fluorination gas is derived from an F₂-electrolysis reactor (fluorine cell), purified or unpurified, of from about 92% by volume of elemental fluorine (F₂) up to about 99% by volume of elemental fluorine (F₂), and most preferably in a very practical range of from about 94% by volume to about 99% by volume; each given range based on the fluorine (F₂) gas and the inert gas as 100% by volume, i.e., based on the total F₂-fluorination gas composition as 100% by volume.

According to a preferred embodiment of the present invention, the compound perfluoro(methylvinylether) (PFMVE) and/or the compound 1,1,2,2-tetrafluoro-1-(trifluoromethoxy)ethane (TFTFME) (E 227), respectively, can also be prepared in a continuous manner. More preferably, the compound perfluoro(methyl vinyl ether) (PFMVE) and/or the compound 1,1,2,2-tetrafluoro-1-(trifluoromethoxy)ethane (TFTFME) (E 227), respectively, in microreactor reaction.

Optionally, any intermediate in the process for manufacturing of perfluoro(methylvinylether) (PFMVE) and/or 1,1,2,2-tetrafluoro-1-(trifluoromethoxy)ethane (TFTFME) (E 227) compound according to the invention may be isolated and/or purified, and then such isolated and/or purified may be further processed, as desired. For example, the compound 1,1,2,2-tetrafluoro-1-(trifluoromethoxy)ethane (TFTFME) (E 227), which is a suitable intermediate in the manufacture of perfluoro(methyl vinyl ether) (PFMVE), may be isolated and/or purified. For example, the compound 1,1,2,2-tetrafluoro-1-(trifluoromethoxy)ethane (TFTFME) (E 227) is prepared in a first microreactor by fluorination (A) is optionally isolated and/or purified, and then the compound 1,1,2,2-tetrafluoro-1-(trifluoromethoxy)ethane (TFTFME) (E 227) is transferred into another (second) microreactor to be further reacted in reaction step (B) for HF-elimination.

The intermediate compound 1,1,2,2-tetrafluoro-1-(trifluoromethoxy)ethane (TFTFME) (E 227) produced in the mentioned first microreactor sequence by fluorination (A) and HF-elimination (B) reaction, optionally may be isolated and/or purified, and then can also constitute the final product in isolated and/or purified form.

Alternatively, (intermediate) compound 1,1,2,2-tetrafluoro-1-(trifluoromethoxy)ethane (TFTFME) (E 227) produced in a first microreactor by fluorination (A) reaction, as a crude compound as obtained (e.g., not further purified), is transferred into the mentioned another (second) microreactor, to be further reacted in the HF-elimination (B) reaction to yield the final target compound perfluoro(methyl vinyl ether) (PFMVE).

In a further variant of the present invention, see for example, Example 2 and reaction Scheme 3, the final target compound perfluoro(methylvinylether) (PFMVE) can also be prepared out of the (intermediate) compound 1,1,2,2-tetrafluoro-1-(trifluoromethoxy)ethane (TFTFME) (E 227), and described herein above in more detail. Preferably, the reaction can be performed in a continuous manner.

Microreactor Process:

The invention also may pertain to a process for manufacturing of perfluoro(methylvinylether) (PFMVE), and/or of 1,1,2,2-tetrafluoro-1-(trifluoromethoxy)ethane (TFTFME) (E 227), which is a suitable intermediate in the manufacture of perfluoro(methylvinylether) (PFMVE), wherein the process is a continuous process, preferably wherein the continuous process is carried out in a microreactor.

The invention may employ more than a single microreactor, i.e., the invention may employ two, three, four, five or more microreactors, for either extending the capacity or residence time, for example, to up to ten microreactors in parallel or four microreactors in series. If more than a single microreactor is employed, then the plurality of microreactors can be arranged either sequentially or in parallel, and if three or more microreactors are employed, these may be arranged sequentially, in parallel or both.

The invention is also very advantageous, in to embodiments wherein the process for manufacturing of perfluoro(methyl vinyl ether) (PFMVE) and/or of 1,1,2,2-tetrafluoro-1-(trifluoromethoxy)ethane (TFTFME) (E 227) according to the invention optionally is performed in a continuous flow reactor system, or preferably in a microreactor system.

In an preferred embodiment the invention relates to a process for manufacturing of perfluoro(methyl vinyl ether) (PFMVE) and/or of 1,1,2,2-tetrafluoro-1-(trifluoromethoxy)ethane (TFTFME) (E 227), wherein in at least one reaction step of (A) and (B) is carried out as a continuous processes, wherein the continuous process is performed in at least one continuous flow reactor with upper lateral dimensions of about ≤5 mm, or of about ≤4 mm,

preferably in at least one microreactor; more preferably wherein of the said at least one reaction step is a continuous process in at least one microreactor under one or more of the following conditions:
flow rate: of from about 10 ml/h up to about 400 l/h;
temperature: of from about 0° C. up to about 150° C.;
pressure: of from about 4 bar up to about 50 bar;
residence time: of from about 1 second, preferably from about 1 minute, up to about 60 minutes.

In another preferred embodiment the invention relates to such a process of preparing a compound according to the invention, wherein at least one of the said continuous flow reactors, preferably at least one of the microreactors, independently is a SiC-continuous flow reactor, preferably independently is a SiC-microreactor.

The Continuous Flow Reactors and Microreactors:

In addition to the above, according to one aspect of the invention, also a plant engineering invention is provided, as used in the process invention and described herein, pertaining to the optional, and in some embodiments of the process invention, the process even preferred implementation in microreactors.

As to the term “microreactor”: A “microreactor” or “microstructured reactor” or “microchannel reactor”, in one embodiment of the invention, is a device in which chemical reactions take place in a confinement with typical lateral dimensions of about ≤1 mm; an example of a typical form of such confinement are microchannels. Generally, in the context of the invention, the term “microreactor”: A “microreactor” or “microstructured reactor” or “microchannel reactor”, denotes a device in which chemical reactions take place in a confinement with typical lateral dimensions of about ≤5 mm.

Microreactors are studied in the field of micro process engineering, together with other devices (such as micro heat exchangers) in which physical processes occur. The microreactor is usually a continuous flow reactor (contrast with/to a batch reactor). Microreactors offer many advantages over conventional scale reactors, including vast improvements in energy efficiency, reaction speed and yield, safety, reliability, scalability, on-site/on-demand production, and a much finer degree of process control.

Microreactors are used in “flow chemistry” to perform chemical reactions.

In flow chemistry, wherein often microreactors are used, a chemical reaction is run in a continuously flowing stream rather than in batch production. Batch production is a technique used in manufacturing, in which the object in question is created stage by stage over a series of workstations, and different batches of products are made. Together with job production (one-off production) and mass production (flow production or continuous production) it is one of the three main production methods. In contrast, in flow chemistry the chemical reaction is run in a continuously flowing stream, wherein pumps move fluid into a tube, and where tubes join one another, the fluids contact one another. If these fluids are reactive, a reaction takes place. Flow chemistry is a well-established technique for use at a large scale when manufacturing large quantities of a given material. However, the term has only been coined recently for its application on a laboratory scale.

Continuous flow reactors, e.g. such as used as microreactor, are typically tube like and manufactured from non-reactive materials, such known in the prior art and depending on the specific purpose and nature of possibly aggressive agents and/or reactants. Mixing methods include diffusion alone, e.g. if the diameter of the reactor is narrow, e.g. <1 mm, such as in microreactors, and static mixers. Continuous flow reactors allow good control over reaction conditions including heat transfer, time and mixing. The residence time of the reagents in the reactor, i.e. the amount of time that the reaction is heated or cooled, is calculated from the volume of the reactor and the flow rate through it: Residence time=Reactor Volume/Flow Rate. Therefore, to achieve a longer residence time, reagents can be pumped more slowly, just a larger volume reactor can be used and/or even several microreactors can be placed in series, optionally just having some cylinders in between for increasing residence time if necessary for completion of reaction steps. In this later case, cyclones after each microreactor help to let escape some low boiling substances, e.g., any formed PFVME together with (potentially present) inert gas and so far to positively influence the reaction performance. Production rates can vary from milliliters per minute to liters per hour.

Some examples of flow reactors are spinning disk reactors (Colin Ramshaw); spinning tube reactors; multi-cell flow reactors; oscillatory flow reactors; microreactors; hex reactors; and aspirator reactors. In an aspirator reactor a pump propels one reagent, which causes a reactant to be sucked in. Also to be mentioned are plug flow reactors and tubular flow reactors.

In the present invention, in one embodiment it is particularly preferred to employ a microreactor.

In the use and processes according to the invention in a preferred embodiment the invention is using a microreactor. But it is to be noted in a more general embodiment of the invention, apart from the said preferred embodiment of the invention that is using a microreactor, any other, e.g. preferentially pipe-like, continuous flow reactor with upper lateral dimensions of up to about 1 cm, and as defined herein, can be employed. Thus, such a continuous flow reactor preferably with upper lateral dimensions of up to about ≤5 mm, or of about ≤4 mm, refers to a preferred embodiment of the invention, e.g. preferably to a microreactor. Continuously operated series of STRs is another option, but less preferred than using a microreactor.

In the before said embodiments of the invention, the minimal lateral dimensions of the, e.g. preferentially pipe-like, continuous flow reactor can be about >5 mm; but is usually not exceeding about 1 cm. Thus, the lateral dimensions of the, e.g. preferentially pipe-like, continuous flow reactor can be in the range of from about >5 mm up to about 1 cm, and can be of any value therein between. For example, the lateral dimensions of the, e.g. preferentially pipe-like, continuous flow reactor can be about 5.1 mm, about 5.5 mm, about 6 mm, about 6.5 mm, about 7 mm, about 7.5 mm, about 8 mm, about 8.5 mm, about 9 mm, about 9.5 mm, and about 10 mm, or can be can be of any value intermediate between the said values.

In the before said embodiments of the invention using a microreactor preferentially the minimal lateral dimensions of the microreactor can be at least about 0.25 mm, and preferably at least about 0.5 mm; but the maximum lateral dimensions of the microreactor does not exceed about ≤5 mm. Thus, the lateral dimensions of the, e.g. preferential microreactor can be in the range of from about 0.25 mm up to about ≤5 mm, and preferably from about 0.5 mm up to about ≤5 mm, and can be of any value therein between. For example, the lateral dimensions of the preferential microreactor can be about 0.25 mm, about 0.3 mm, about 0.35 mm, about 0.4 mm, about 0.45 mm, and about 5 mm, or can be can be of any value intermediate between the said values.

As stated here before in the embodiments of the invention in its broadest meaning is employing, preferentially pipe-like, continuous flow reactor with upper lateral dimensions of up to about 1 cm. Such continuous flow reactor, for example is a plug flow reactor (PFR).

The plug flow reactor (PFR), sometimes called continuous tubular reactor, CTR, or piston flow reactors, is a reactor used to perform and describe chemical reactions in continuous, flowing systems of cylindrical geometry. The PFR reactor model is used to predict the behavior of chemical reactors of such design, so that key reactor variables, such as the dimensions of the reactor, can be estimated.

Fluid going through a PFR may be modeled as flowing through the reactor as a series of infinitely thin coherent “plugs”, each with a uniform composition, traveling in the axial direction of the reactor, with each plug having a different composition from the ones before and after it. The key assumption is that as a plug flows through a PFR, the fluid is perfectly mixed in the radial direction (i.e. in the lateral direction) but not in the axial direction (forwards or backwards).

Accordingly, the terms used herein to define the reactor type used in the context of the invention such like “continuous flow reactor”, “plug flow reactor”, “tubular reactor”, “continuous flow reactor system”, “plug flow reactor system”, “tubular reactor system”, “continuous flow system”, “plug flow system”, “tubular system” are synonymous to each other and interchangeably by each other.

The reactor or system may be arranged as a multitude of tubes, which may be, for example, linear, looped, meandering, circled, coiled, or combinations thereof. If coiled, for example, then the reactor or system is also called “coiled reactor” or “coiled system”.

In the radial direction, i.e. in the lateral direction, such reactor or system may have an inner diameter or an inner cross-section dimension (i.e. radial dimension or lateral dimension, respectively) of up to about 1 cm. Thus, in an embodiment the lateral dimension of the reactor or system may be in the range of from about 0.25 mm up to about 1 cm, preferably of from about 0.5 mm up to about 1 cm, and more preferably of from about 1 mm up to about 1 cm.

In further embodiments the lateral dimension of the reactor or system may be in the range of from about >5 mm to about 1 cm, or of from about 5.1 mm to about 1 cm.

If the lateral dimension at maximum of up to about ≤5 mm, or of up to about ≤4 mm, then the reactor is called “microreactor”. Thus, in still further microreactor embodiments the lateral dimension of the reactor or system may be in the range of from about 0.25 mm up to about ≤5 mm, preferably of from about 0.5 mm up to about ≤5 mm, and more preferably of from about 1 mm up to about ≤5 mm; or the lateral dimension of the reactor or system may be in the range of from about 0.25 mm up to about ≤4 mm, preferably of from about 0.5 mm up to about ≤4 mm, and more preferably of from about 1 mm up to about ≤4 mm.

In an alternative embodiment of the invention, it is also optionally desired to employ another continuous flow reactor than a microreactor, preferably if, for example, the (halogenation promoting, e.g. the halogenation or preferably the halogenation) catalyst composition used in the halogenation or fluorination tends to get viscous during reaction or is viscous already as a said catalyst as such. In such case, a continuous flow reactor, i.e. a device in which chemical reactions take place in a confinement with lower lateral dimensions of greater than that indicated above for a microreactor, i.e. of greater than about 1 mm, but wherein the upper lateral dimensions are about ≤4 mm. Accordingly, in this alternative embodiment of the invention, employing a continuous flow reactor, the term “continuous flow reactor” preferably denotes a device in which chemical reactions take place in a confinement with typical lateral dimensions of from about ≥1 mm up to about ≤4 mm. In such an embodiment of the invention it is particularly preferred to employ as a continuous flow reactor a plug flow reactor and/or a tubular flow reactor, with the said lateral dimensions. Also, in such an embodiment of the invention, as compared to the embodiment employing a microreactor, it is particularly preferred to employ higher flow rates in the continuous flow reactor, preferably in the plug flow reactor and/or a tubular flow reactor, with the said lateral dimensions. For example, such higher flow rates, are up to about 2 times higher, up to about 3 times higher, up to about 4 times higher, up to about 5 times higher, up to about 6 times higher, up to about 7 times higher, or any intermediate flow rate of from about ≥1 up to about ≤7 times higher, of from about ≥1 up to about ≤6 times higher, of from about ≥1 up to about ≤5 times higher, of from about ≥1 up to about ≤4 times higher, of from about ≥1 up to about ≤3 times higher, or of from about ≥1 up to about ≤2 times higher, each as compared to the typical flow rates indicated herein for a microreactor. Preferably, the said continuous flow reactor, more preferably the plug flow reactor and/or a tubular flow reactor, employed in this embodiment of the invention is configured with the construction materials as defined herein for the microreactors. For example, such construction materials are silicon carbide (SiC) and/or are alloys such as a highly corrosion resistant nickel-chromium-molybdenum-tungsten alloy, e.g. Hastelloy®, as described herein for the microreactors.

A very particular advantage of the present invention employing a microreactor, or a continuous flow reactor with the before said lateral dimensions, the number of separating steps can be reduced and simplified, and may be devoid of time and energy consuming, e.g. intermediate, distillation steps. Especially, it is a particular advantage of the present invention employing a microreactor, or a continuous flow reactor with the before said lateral dimensions, that for separating simply phase separation methods can be employed, and the non-consumed reaction components may be recycled into the process, or otherwise be used as a product itself, as applicable or desired.

In addition to the preferred embodiments of the present invention using a microreactor according to the invention, in addition or alternatively to using a microreactor, it is also possible to employ a plug flow reactor or a tubular flow reactor, respectively.

Plug flow reactor or tubular flow reactor, respectively, and their operation conditions, are well known to those skilled in the field.

Although the use of a continuous flow reactor with upper lateral dimensions of about ≤5 mm, or of about ≤4 mm, respectively, and in particular of a microreactor, is particularly preferred in the present invention, depending on the circumstances, it could be imagined that somebody dispenses with an microreactor, then of course with yield losses and higher residence time, higher temperature, and instead takes a plug flow reactor or turbulent flow reactor, respectively. However, this could have a potential advantage, taking note of the mentioned possibly disadvantageous yield losses, namely the advantage that the probability of possible blockages (tar particle formation by non-ideal driving style) could be reduced because the diameters of the tubes or channels of a plug flow reactor are greater than those of a microreactor.

The possibly allegeable disadvantage of this variant using a plug flow reactor or a tubular flow reactor, however, may also be seen only as subjective point of view, but on the other hand under certain process constraints in a region or at a production facility may still be appropriate, and loss of yields be considered of less importance or even being acceptable in view of other advantages or avoidance of constraints.

In the following, the invention is more particularly described in the context of using a microreactor. Preferentially, a microreactor used according to the invention is a ceramic continuous flow reactor, more preferably an SiC (silicon carbide) continuous flow reactor, and can be used for material production at a multi-to scale. Within integrated heat exchangers and SiC materials of construction, it gives optimal control of challenging flow chemistry application. The compact, modular construction of the flow production reactor enables, advantageously for: long term flexibility towards different process types; access to a range of production volumes (5 to 400 l/h); intensified chemical production where space is limited; unrivalled chemical compatibility and thermal control.

Ceramic (SiC) microreactors, are e.g. advantageously diffusion bonded 3M SiC reactors, especially braze and metal free, provide for excellent heat and mass transfer, superior chemical compatibility, of FDA certified materials of construction, or of other drug regulatory authority (e.g. EMA) certified materials of construction. Silicon carbide (SiC), also known as carborundum, is a containing silicon and carbon, and is well known to those skilled in the art. For example, synthetic SiC powder is been mass-produced and processed for many technical applications.

For example, in the embodiments of the invention the objects are achieved by a method in which at least one reaction step takes place in a microreactor. Particularly, in preferred embodiments of the invention the objects are achieved by a method in which at least one reaction step takes place in a microreactor that is comprising or is made of SiC (“SiC-microreactor”), or in a microreactor that is comprising or is made of an alloy, e.g. such as Hastelloy C, as it is each defined herein after in more detail.

Preferred Hastelloy C4 nickel alloys are already described further above. See, for example, Table 1.

Thus, without being limited to, for example, in an embodiment of the invention the microreactor suitable for, preferably for industrial, production an “SiC-microreactor” that is comprising or is made of SiC (silicon carbide; e.g. SiC as offered by Dow Corning as Type G1SiC or by Chemtrix MR555 Plantrix), e.g. providing a production capacity of from about 5 up to about 400 kg per hour; or without being limited to, for example, in another embodiment of the invention the microreactor suitable for industrial production is comprising or is made of Hastelloy C, as offered by Ehrfeld. Such microreactors are particularly suitable for the, preferably industrial, production of fluorinated products according to the invention.

In order to meet both the mechanical and chemical demands placed on production scale flow reactors, Plantrix modules are fabricated from 3M™SiC (Grade C). Produced using the patented 3M (EP 1 637 271 Bi and foreign patents) diffusion bonding technology, the resulting monolithic reactors are hermetically sealed and are free from welding lines/joints and brazing agents. More technical information on the Chemtrix MR555 Plantrix can be found in the brochure “CHEMTRIX—Scalable Flow Chemistry—Technical Information Plantrix® MR555 Series, published by Chemtrix BV in 2017, which technical information is incorporated herein by reference in its entirety.

Apart from the before said example, in other embodiments of the invention, in general SiC from other manufactures, and as known to the skilled person, of course can be employed in the present invention.

Accordingly, in the present invention as microreactor also the Protrix® of by Chemtrix can be used. Protrix® is a modular, continuous flow reactor fabricated from 3M® silicon carbide, offering superior chemical resistance and heat transfer. In order to meet both the mechanical and chemical demands placed on flow reactors, Protrix® modules are fabricated from 3M® SiC (Grade C). Produced using the patented 3M (EP 1 637 271 Bi and foreign patents) diffusion bonding technology, the resulting monolithic reactors are hermetically sealed and are free from welding lines/joints and brazing agents. This fabrication technique is a production method that gives solid SiC reactors (thermal expansion coefficient=4.1×10⁻⁶K⁻¹).

Designed for flow rates ranging from 0.2 to 20 ml/min and pressures up to 25 bar, Protrix® allows the user to develop continuous flow processes at the lab-scale, later transitioning to Plantrix® MR555 (×340 scale factor) for material production. The Protrix® reactor is a unique flow reactor with the following advantages: diffusion bonded 3M® SiC modules with integrated heat exchangers that offer unrivaled thermal control and superior chemical resistance; safe employment of extreme reaction conditions on a g scale in a standard fume hood; efficient, flexible production in terms of number of reagent inputs, capacity or reaction time. The general specifications for the Protrix® flow reactors are summarized as follows; possible reaction types are, e.g. A+B→P1+Q (or C)→P, wherein the terms “A”, “B” and “C” represent educts, “P” and “P1” products, and “Q” quencher; throughput (ml/min) of from about 0.2 up to about 20; channel dimensions (mm) of 1×1 (pre-heat and mixer zone), 1.4×1.4 (residence channel); reagent feeds of 1 to 3; module dimensions (width×height) (mm) of 110×260; frame dimensions (width×height×length) (mm) approximately 400×300×250; number of modules/frame is one (minimum) up to four (max.). More technical information on the ChemtrixProtrix® reactor can be found in the brochure “CHEMTRIX—Scalable Flow Chemistry—Technical Information Protrix®, published by Chemtrix BV in 2017, which technical information is incorporated herein by reference in its entirety.

The Dow Corning as Type G1SiC microreactor, which is scalable for industrial production, and as well suitable for process development and small production can be characterized in terms of dimensions as follows: typical reactor size (length×width×height) of 88 cm×38 cm×72 cm; typical fluidic module size of 188 mm×162 mm. The features of the Dow Corning as Type G1SiC microreactor can be summarized as follows: outstanding mixing and heat exchange: patented HEART design; small internal volume; high residence time; highly flexible and multipurpose; high chemical durability which makes it suitable for high pH compounds and especially hydrofluoric acid; hybrid glass/SiC solution for construction material; seamless scale-up with other advanced-flow reactors. Typical specifications of the Dow Corning as Type G1SiC microreactor are as follows: flow rate of from about 30 ml/min up to about 200 ml/min; operating temperature in the range of from about −60° C. up to about 200° C., operating pressure up to about 18 barg (“barg” is a unit of gauge pressure, i.e. pressure in bars above ambient or atmospheric pressure); materials used are silicon carbide, PFA (perfluoroalkoxy alkanes), perfluoroelastomer; fluidic module of 10 ml internal volume; options: regulatory authority certifications, e.g. FDA or EMA, respectively. The reactor configuration of Dow Corning as Type G1SiC microreactor is characterized as multipurpose and configuration can be customized. Injection points may be added anywhere on the said reactor.

Hastelloy® C is an alloy represented by the formula NiCr21Mol4W, alternatively also known as “alloy 22” or “Hastelloy® C-22. The said alloy is well known as a highly corrosion resistant nickel-chromium-molybdenum-tungsten alloy and has excellent resistance to oxidizing reducing and mixed acids. The said alloy is used in flue gas desulphurization plants, in the chemical industry, environmental protection systems, waste incineration plants, sewage plants. Apart from the before said example, in other embodiments of the invention, in general nickel-chromium-molybdenum-tungsten alloy from other manufactures, and as known to the skilled person, of course can be employed in the present invention. A typical chemical composition (all in weight-%) of such nickel-chromium-molybdenum-tungsten alloy is, each percentage based on the total alloy composition as 100%: Ni (nickel) as the main component (balance) of at least about 51.0%, e.g. in a range of from about 51.0% to about 63.0%; Cr (chromium) in a range of from about 20.0 to about 22.5%, Mo (molybdenum) in a range of from about 12.5 to about 14.5%, W (tungsten or wolfram, respectively) in a range of from about 2.5 to about 3.5%; and Fe (iron) in an amount of up to about 6.0%, e.g. in a range of from about 1.0% to about 6.0%, preferably in a range of from about 1.5% to about 6.0%, more preferably in a range of from about 2.0% to about 6.0%. Optionally, the percentage based on the total alloy composition as 100%, Co (cobalt) can be present in the alloy in an amount of up to about 2.5%, e.g. in a range of from about 0.1% to about 2.5%. Optionally, the percentage based on the total alloy composition as 100%, V (vanadium) can be present in the alloy in an amount of up to about 0.35%, e.g. in a range of from about 0.1% to about 0,35%. Also, the percentage based on the total alloy composition as 100%, optionally low amounts (i.e. ≤0.1%) of other element traces, e.g. independently of C (carbon), Si (silicon), Mn (manganese), P (phosphor), and/or S (sulfur). In such case of low amounts (i.e. ≤0.1%) of other elements, the said elements e.g. of C (carbon), Si (silicon), Mn (manganese), P (phosphor), and/or S (sulfur), the percentage based on the total alloy composition as 100%, each independently can be present in an amount of up to about 0.1%, e.g. each independently in a range of from about 0.01 to about 0.1%, preferably each independently in an amount of up to about 0.08%, e.g. each independently in a range of from about 0.01 to about 0.08%. For example, said elements e.g. of C (carbon), Si (silicon), Mn (manganese), P (phosphor), and/or S (sulfur), the percentage based on the total alloy composition as 100%, each independently can be present in an amount of, each value as an about value: C≤0.01%, Si 0.08%, Mn≤0.05%, P≤0.015%, S≤0.02%. Normally, no traceable amounts of any of the following elements are found in the alloy compositions indicated above: Nb (niobium), Ti (titanium), Al (aluminum), Cu (copper), N (nitrogen), and Ce (cerium).

Hastelloy® C-276 alloy was the first wrought, nickel-chromium-molybdenum material to alleviate concerns over welding (by virtue of extremely low carbon and silicon contents). As such, it was widely accepted in the chemical process and associated industries, and now has a 50-year-old track record of proven performance in a vast number of corrosive chemicals. Like other nickel alloys, it is ductile, easy to form and weld, and possesses exceptional resistance to stress corrosion cracking in chloride-bearing solutions (a form of degradation to which the austenitic stainless steels are prone). With its high chromium and molybdenum contents, it is able to withstand both oxidizing and non-oxidizing acids, and exhibits outstanding resistance to pitting and crevice attack in the presence of chlorides and other halides. The nominal composition in weight-% is, based on the total composition as 100%: Ni (nickel) 57% (balance); Co (cobalt) 2.5% (max.); Cr (chromium) 16%; Mo (molybdenum) 16%; Fe (iron) 5%; W (tungsten or wolfram, respectively) 4%; further components in lower amounts can be Mn (manganese) up to 1% (max.); V (vanadium) up to 0.35% (max.); Si (silicon) up to 0.08% (max.); C (carbon) 0.01 (max.); Cu (copper) up to 0.5% (max.).

In another embodiments of the invention, without being limited to, for example, the microreactor suitable for the said production, preferably for the said industrial production, is an SiC-microreactor that is comprising or is made only of SiC as the construction material (silicon carbide; e.g. SiC as offered by Dow Corning as Type G1SiC or by Chemtrix MR555 Plantrix), e.g. providing a production capacity of from about 5 up to about 400 kg per hour.

It is of course possible according to the invention to use one or more microreactors, preferably one or more SiC-microreactors, in the production, preferably in the industrial production, of the fluorinated products according to the invention. If more than one microreactor, preferably more than one SiC-microreactor, are used in the production, preferably in the industrial production, of the fluorinated products according to the invention, then these microreactors, preferably these SiC-microreactors, can be used in parallel and/or subsequent arrangements. For example, two, three, four, or more microreactors, preferably two, three, four, or more SiC-microreactors, can be used in parallel and/or subsequent arrangements.

For laboratory search, e.g. on applicable reaction and/or upscaling conditions, without being limited to, for example, as a microreactor the reactor type Plantrix of the company Chemtrix is suitable. Sometimes, if gaskets of a microreactor are made out of other material than HDPTFE, leakage might occur quite soon after short time of operation because of some swelling, so HDPTFE gaskets secure long operating time of microreactor and involved other equipment parts like settler and distillation columns.

For example, an industrial flow reactor (“IFR”, e.g. Plantrix® MR555) comprises of SiC modules (e.g. 3M® SiC) housed within a (non-wetted) stainless steel frame, through which connection of feed lines and service media are made using standard Swagelok fittings. The process fluids are heated or cooled within the modules using integrated heat exchangers, when used in conjunction with a service medium (thermal fluid or steam), and reacted in zig-zag or double zig-zag, meso-channel structures that are designed to give plug flow and have a high heat exchange capacity. A basic IFR (e.g. Plantrix® MR555) system comprises of one SiC module (e.g. 3M® SiC), a mixer (“MRX”) that affords access to A+B→P type reactions. Increasing the number of modules leads to increased reaction times and/or system productivity. The addition of a quench Q/C module extends reaction types to A+B→P1+Q (or C)→P and a blanking plate gives two temperature zones. Herein the terms “A”, “B” and “C” represent educts, “P” and “P1” products, and “Q” quencher.

Typical dimensions of an industrial flow reactor (“IFR”, e.g. Plantrix® MR555) are, for example: channel dimensions in (mm) of 4×4 (“MRX”, mixer) and 5×5 (MRH-I/MRH-II; “MR” denotes residence module); module dimensions (width×height) of 200 mm×555 mm; frame dimensions (width×height) of 322 mm×811 mm. A typical throughput of an industrial flow reactor (“IFR”, e.g. Plantrix® MR555) is, for example, in the range of from about 50 l/h to about 400 l/h. in addition, depending on fluid properties and process conditions used, the throughput of an industrial flow reactor (“IFR”, e.g. Plantrix® MR555), for example, can also be >400 l/h. The residence modules can be placed in series in order to deliver the required reaction volume or productivity. The number of modules that can be placed in series depends on the fluid properties and targeted flow rate.

Typical operating or process conditions of an industrial flow reactor (“IFR”, e.g. Plantrix® MR555) are, for example: temperature range of from about −30° C. to about 200° C.; temperature difference (service—process)<70° C.; reagent feeds of 1 to 3; maximum operating pressure (service fluid) of about 5 bar at a temperature of about 200° C.; maximum operating pressure (process fluid) of about 25 bar at a temperature of about ≤200° C.

The following examples are intended to further illustrate the invention without limiting its scope.

### EXAMPLES

The following examples are intended to further illustrate the invention without limiting its scope.

### Example 1

Fluorination of HFE-254 to E 227 (TFTFME) in a counter-current system with diluted F₂-gas (first step), and base initiated HF-elimination from E 227 (TFTFME) to obtain PFMVE (second step).

Apparatus:

A column made out of Hastelloy C4 with a length of 30 cm and with HDPTFE fillings and a diameter of 5 cm was used according to the drawing below. The liquid reservoir had a volume of 2 l. The pump was a centrifugal pump from company Schmitt. A pressure valve on top of the tower was installed to regulate the pressure. A heat exchanger for heating and cooling was installed into the loop as drawn in FIG. 1.

### Example 1a (First Step)

Selective direct fluorination of HFE-254 to E 227 (TFTFME).

The reservoir was filled with 1000 g (7.57 mol) HFE-254 (1,1,2,2-tetrafluoro-1-(methoxy)ethane) and the pump was started (flow of about 1500 l/h). 10% F₂-gas (dilution in N₂) was fed over a Bronkhorst mass flow meter into the tower so that the reaction temperature was kept at 30° C. while the pressure on the tower was kept at 10 bar abs. by the pressure valve. After 1 h 893 g (23.5 mol) F₂ (10% F₂ in N₂ as inert gas) were fed into the system while the inert N₂ together with some traces of formed HF and traces of E 227 left the apparatus over the pressure valve over the top into an efficient scrubber. After 10 min of further looping without any dosage, the pump was stopped. A sample was taken with a high grade stainless steel cylinder out of the reservoir to which after stopping of the pump all material has fallen down. This reservoir now is containing mainly the product E 227 (TFTFME) (as intermediate or final product) and most of the HF formed in the fluorination reaction (some of the HF may already have escaped together with the inert gas). The material in the cylinder carefully was poured into ice water into another pressure vessel (volume 2 l) and shaken for 5 min to remove HF into the water phase, the GC-analysis (GC=gas chromatography) of a vaporized organic phase showed an E 227 (TFTFME) concentration of 96%.

The phases were separated and the organic phase (not further dried) containing the E 227 (TFTFME) as an intermediate product was re-introduced into the countercurrent system for the next step. See Example 1b.

Alternatively, the organic phase containing the E 227 (TFTFME) was further worked up and optionally further purified to yield an isolated and/or further purified E 227 (TFTFME) as the final product. See Example 3.

### Example 1b (Second Step)

Base initiated HF-elimination from E 227 (TFTFME) to obtain PFMVE.

In the next step, the pump was restarted and 921 g (9.1 mol) NEt₃ were fed before the heat exchanger into the looping reaction mixture by using a piston dosage pump.

Remark on the quantity (9.1 mol) of NEt₃ used in this Example: Since three mol of HF formed in the fluorination step have already been removed with the ice water in Example 1a, based on the quantity of 1000 g (7.57 mol) HFE-254), per se only 7.57 mol of NEt₃ should be necessary in view of (1:1) stoichiometry for the HF-elimination step. As NEt₃ even can take up three HF (complex formation) theoretically only 2.52 mol NEt₃ would be necessary instead of 7.57 mol NEt₃ stoichiometry (1:1). Since here, however, a phase separation was desired, the base NEt₃ was used in high excess NEt₃ as compared to (1:1) stoichiometry.

For this second step, the pressure was reduced to 7 bar abs. and the NEt₃ feed was adjusted such that the temperature of the exothermic elimination reaction did not exceed 40° C. After 40 min, all NEt₃ was fed in. After 10 min of further looping without any dosage, the Schmitt pump was stopped and after further 10 min (temperature of the mixture has reached 20° C.), a second phase was observed, analysis of the lower phase indicated a PFMVE concentration of 96% which after distillation in a 20 cm Vigreux column gave 1.17 kg PFMVE (corresponding to a yield of 93%) with a purity of 98.6% (GC). The sample for GC was taken into a gas mouse and injected into the GC as gas sample (GC column: 50 m Angilent CP-SIL 8)

### Example 2

Synthesis of PFMVE by treatment of (crude) E 227 (TFTFME) with NEt₃.

In this Example 2 a base initiated HF-elimination from E 227 (TFTFME) is performed to obtain PFMVE, and the PFMVE is further isolated and purified by distillation.

The compound E 227 (TFTFME) raw material was prepared in a countercurrent system as described in Example 1a, but instead of work up with ice water, the material in the reservoir after fluorination (containing E 227 and the formed HF) was transferred into a pressure distillation column made out of Hastelloy C4, and which was equipped with a condenser. Then, the raw material containing the crude E 227 carefully treated with a slow feed of NEt₃ (11 mol) at maximum temperature of 40° C. until no exothermic activity could be observed anymore. The product E 227 finally was distilled off at 5 bar abs., and at a transition temperature of −1° C. to yield 1,029 g (82%) PFMVE as yellow liquid.

Remark on the quantity (11 mol) of NEt₃ used in this Example: for four mol HF to be consumed, in view of (1:1) stoichiometry a quantity of 30.28 mol of NEt₃ should be necessary. Since here, in this Example no phase separation was to be performed a quantity of only 11 mol of NEt₃ was sufficient to be used, corresponding to a 10% excess of base NEt₃. Alternatively, of course, this treatment and distillation described in this Example 2, instead of treating crude E 227 still containing HF, can also be performed with isolated and purified E 227 (i.e., E 227 not containing HF anymore).

### Example 3

Isolation and further purification of E 227 (TFTFME) by distillation.

The compound E 227 (TFTFME) raw material was prepared in a countercurrent system as described in Example 1a. After the work up with ice water, the organic phase containing the crude compound E 227 (TFTFME) was dried for 30 min over Na₂SO₄. Then, the dried organic phase containing the crude compound E 227 (TFTFME) was transferred into a pressure distillation column made out of Hastelloy C4, and which was equipped with a condenser and kept at a temperature of −20° C.

The product E 227 finally was distilled off at 5 bar abs., and at a transition temperature of 18° C. to yield 1,310 g (93%) with a purity of 98.4% (GC)

### Example 4

Continuous conversion of HFC 254 to PFMVE in a two-step microreactor system.

Apparatus:

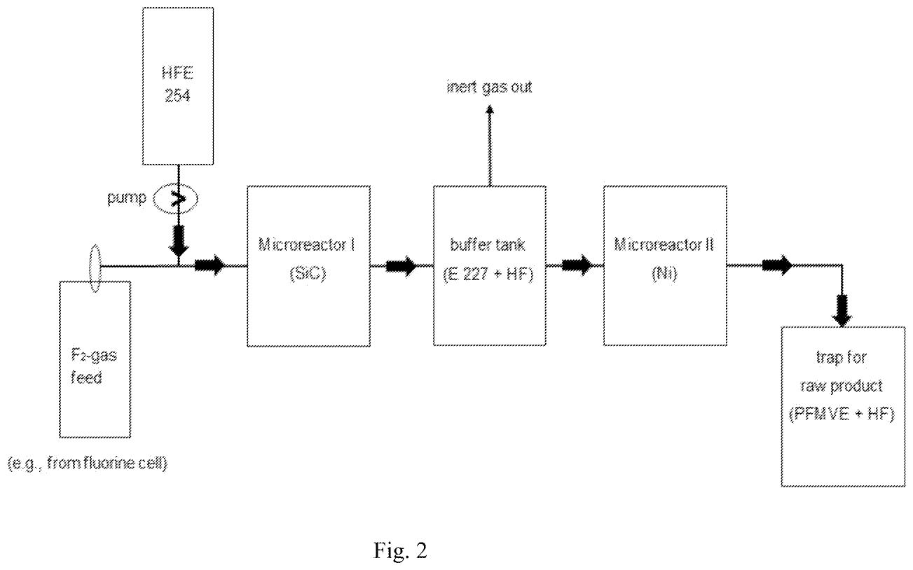

For the first step (fluorination) a 27 ml microreactor (microreactor I) made out of SiC (silicium carbide) was used. For the elimination step, a second microreactor (microreactor II) made out of Ni (nickel) with a volume of 54 ml was installed in series; the first microreactor was kept at room temperature (ambient temperature, e.g., about 25° C.) by cooling, the second microreactor was heated to 80° C. After the first microreactor, there is a cyclone (not shown in the FIG. 2) allowing some inert gas to leave the system over a pressure valve installed at the cyclone. A buffer tank with filling level measurement is also installed between first and second microreactor allowing regulating and balancing the feed between the reactors by a Swagelok hand valve. There is a cooler installed after the second microreactor (also not shown in the FIG. 2) to cool down the reaction mixture to 0° C. which is fed after microreactor II into the cooled trap (kept at −40° C.) over a deep pipe (the trap is a stainless steel cylinder with deep pipe, gas outlet and pressure valve at gas outlet).

### Example 4a (First Step)

Selective direct fluorination of HFE-254 to E 227 (TFTFME).

Reaction: Before start of reaction, the system is continuously floated with a Nitrogen inert gas purge which was fast reduced to about 5 vol % (vs. F₂) once the feeding of raw materials has started. A fast reduction of inert gas feed is essential as inert gas reduces sharply the heat exchange efficiency in the microchannel reactors. Into this installation F₂ was fed directly out of a Fluorine electrolysis cell over a Bronkhorst mass flow controller together with 150 g (1.14 mol) liquid HFC 254 per hour (h), the pressure in the first microreactor was adjusted to 7 bar abs. at the pressure valve. The liquid phase obtained in the buffer tank contained the E 227 (TFTFME) and HF.

### Example 4b (Second Step)

Ni-catalyzed HF-elimination from E 227 (TFTFME) to obtain PFMVE.

The liquid phase obtained in Example 4a containing the product E 227 (TFTFME) was heated to 80° C. in the second microreactor (made out of Ni) for performing the final HF-elimination which was done at 5 bar abs. to result in PFMVE which was collected in the trap at −30° C. together with the HF formed. Final distillation of PFMVE was done in a pressure column at 5 bar abs. made out of Hastelloy C4 yielding 89% of PFMVE (99.9% GC-purity) as light boiling substance based on HFC 254 starting material, and leaving the HF as bottom product in the column.

### Example 5

Continuous conversion of HFC 254 to PFMVE in a two-step microreactor system, and quenching with NEt₃ (base initiated HF-elimination).

The first reaction step of selective direct fluorination of HFC 254 to yield product E 227 (TFTFME) in HF (as intermediate or final product) was performed as in Example 4. But for the second reaction step, i.e., the quenching or base initiated HF-elimination, the organic base NEt₃ (triethylamine) was fed into the reaction after the buffer tank before the second microreactor (microreactor II) in 1.33 equivalent amounts vs. HFC 254 in the first step (each NEt₃ scavenges 3 equivalents HF), and the thermostat at the second microreactor was switched from heating now to cooling to a temperature of 20° C. in the second microreactor. Pressure was adjusted to 5 bar abs. using a pressure valve at the gas exit of the trap (kept at −30° C.). Efficient cooling is necessary as HF-quenching and HF-elimination with NEt₃ as base is an exothermic process. A second phase was formed immediately in the trap, and in which the lower phase contained the product PFMVE. The product PFMVE was obtained without any further purification necessary, and with a purity of 97.9% (GC) and 95% yield.

### Example 6

Continuous conversion of HFC 254 to PFMVE in a two-step microreactor system, and quenching with NBu₃ (base initiated HF-elimination).

Example 5 was repeated but using as the organic base NBu₃ (tributylamine) instead of NEt₃. The phase separation took up to 1 h, and the crude PFMVE phase (94% purity) also contained some amine compounds.

Final purification of the crude PFMVE phase was done by distillation in a short Vigreux column at 5 bar abs. (like described in Example 4) yielding 82% PFMVE as the product.

What is claimed is:

1. A process for the manufacture of the compound PFMVE (perfluoro(methyl vinyl ether)) having the formula (I), 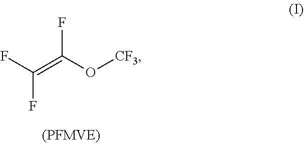
wherein the process comprises the steps of a direct fluorination reaction (A) and HF-elimination reaction (B), in a reactor or reactor system, resistant to elemental fluorine (F2) and hydrogen fluoride (HF):
(A) in a first reaction step a direct fluorination by reacting the compound HFE-254 (1,1,2,2-tetrafluoro-1-(methoxy)ethane) of formula (III), 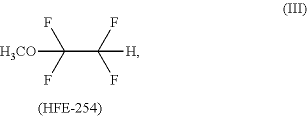
with an about stoichiometric quantity of elemental fluorine (F2) comprised in a fluorination gas to selectively substitute in the compound of formula (III) the three hydrogen atoms of the 1-(methoxy) group of the compound HFE-254 of formula (III) for fluorine, and wherein the reaction is carried out at temperature in the range of from about 0° C. to about +60° C.
and at a pressure in the range of from about 1 bar absolute bar to about 20 bar absolute
to yield the compound TFTFME (1,1,2,2-tetrafluoro-1-(trifluoromethoxy)ethane), (E 227), of formula (II), 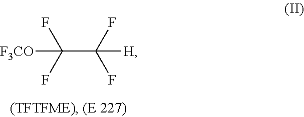
and
with or without isolating and/or purifying the (intermediate) fluorination product (TFTFME), (E 227); preferably without isolating and/or purifying the (intermediate) fluorination product (TFTFME), (E 227),
(B) in a second reaction step an elimination reaction, wherein HF (hydrogen fluoride) is eliminated from the (intermediate) fluorination product (TFTFME), (E 227), of formula (II), obtained in step (A), and the elimination reaction is performed
(i) as a(n) (exothermic) elimination reaction in the presence of one or more nitrogen-containing organic bases,
and/or
in the presence of one or more inorganic bases,
wherein the temperature of the (exothermic) elimination reaction is controlled to not exceed a temperature of about 60° C.,
and wherein the (exothermic) elimination reaction is carried out at a pressure in the range of from about 1 bar absolute bar to about 20 bar absolute
or
(ii) as a, non-catalytic or preferably catalytic, more preferably Ni (nickel) catalytic, thermal elimination reaction at a temperature in the range of about 60° C. to about 120° C.,
to yield the compound PFMVE (perfluoro(methyl vinyl ether)) of formula (I), and
(C) withdrawing and collecting the compound PFMVE (perfluoro(methyl vinyl ether)) of formula (I) obtained in step (B) from the reactor or reactor system,
and
(D) optionally isolating and/or purifying the compound PFMVE (perfluoro(methyl vinyl ether)) of formula (I).

2. A process for the manufacture of the compound PFMVE (perfluoro(methyl vinyl ether)) having the formula (I), 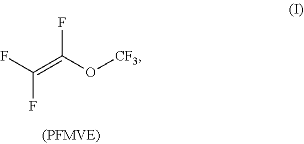
wherein the process comprises, in a reactor or reactor system, resistant to elemental fluorine (F₂) and hydrogen fluoride (HF), performing an HF-elimination reaction step (B), wherein in the elimination reaction, HF (hydrogen fluoride) is eliminated from the compound TFTFME (1,1,2,2-tetrafluoro-1-(trifluoromethoxy)ethane), (E 227), of formula (II), 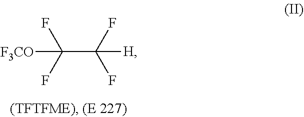
and the elimination reaction step (B) is performed
(i) as a(n) (exothermic) elimination reaction in the presence of one or more nitrogen-containing organic bases,
and/or
in the presence of one or more inorganic bases,
wherein the temperature of the (exothermic) elimination reaction is controlled to not exceed a temperature of about 60° C.,
and wherein the (exothermic) elimination reaction is carried out at a pressure in the range of from about 1 bar absolute bar to about 20 bar absolute,
or
(ii) as a, non-catalytic or preferably catalytic, more preferably Ni (nickel) catalytic, thermal elimination reaction at a temperature in the range of about 60° C. to about 120° C.,
to yield the compound PFMVE (perfluoro(methyl vinyl ether)) of formula (I),
and
(C) withdrawing and collecting the compound PFMVE (perfluoro(methyl vinyl ether)) of formula (I) obtained in step (B) from the reactor or reactor system,
and
(D) optionally isolating and/or purifying the compound PFMVE (perfluoro(methyl vinyl ether)) of formula (I).

3. A process for the manufacture of the compound TFTFME (1,1,2,2-tetrafluoro-1-(trifluoromethoxy)ethane), (E 227), of formula (II), 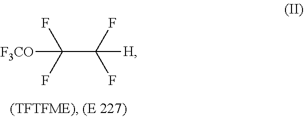
wherein the process comprises, in a reactor or reactor system, resistant to elemental fluorine (F₂) and hydrogen fluoride (HF), performing a direct fluorination reaction step (A), wherein in the direct fluorination reaction the compound HFE-254 (1,1,2,2-tetrafluoro-1-(methoxy)ethane) of formula (III), 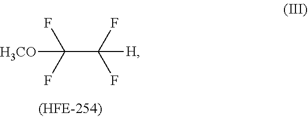
is fluorinated with an about stoichiometric quantity of elemental fluorine (F₂) comprised in a fluorination gas to selectively substitute in the compound of formula (III) the three hydrogen atoms of the 1-(methoxy) group of the compound HFE-254 of formula (III) for fluorine, and wherein the reaction is carried out at temperature in the range of from about 0° C. to about +60° C., and at a pressure in the range of from about 1 bar absolute bar to about 20 bar absolute,
to yield the compound TFTFME (1,1,2,2-tetrafluoro-1-(trifluoromethoxy)ethane), (E 227), of formula (II), 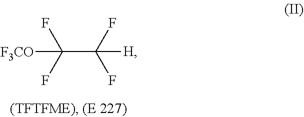
and
(C) withdrawing and collecting the compound TFTFME (1,1,2,2-tetrafluoro-1-(trifluoromethoxy)ethane), (E 227), of formula (II) obtained in step (A) from the reactor or reactor system,
and
(D) optionally isolating and/or purifying the compound TFTFME (1,1,2,2-tetrafluoro-1-(trifluoromethoxy)ethane), (E 227), of formula (II).

4. The process according to claim 1 for the manufacture of the compound PFMVE (perfluoro(methyl vinyl ether)) having the formula (I), wherein the fluorination (A) reaction is performed in a counter-current reactor system, in particular in a loop reactor system, or in a counter-current (loop) system (“inverse gas scrubber system”), and
wherein the fluorine (F₂) concentration in the F₂-fluorination gas is in a range of from about 1% by volume of elemental fluorine (F₂) up to about almost 100% by volume of elemental fluorine (F₂), based on the total F₂-fluorination gas composition as 100% by volume;
preferably wherein
(i) the fluorine (F₂) concentration in the F₂-fluorination gas is in a range of from about 1% by volume of elemental fluorine (F₂) up to about 30% by volume of elemental fluorine (F₂), more preferably of from about 5% by volume of elemental fluorine (F₂) up to about 25% by volume of elemental fluorine (F₂), even more preferably of from about 5% by volume of elemental fluorine (F₂) up to about 20% by volume of elemental fluorine (F₂), each range based on the total F₂-fluorination gas composition as 100% by volume; or
(ii) the fluorine (F₂) concentration in the F₂-fluorination gas is in a range of from about 85% by volume of elemental fluorine (F₂) up to about almost 100% by volume of elemental fluorine (F₂), more preferably of from about 90% by volume of elemental fluorine (F₂) up to about almost 100% by volume of elemental fluorine (F₂), based on the total F₂-fluorination gas composition as 100% by volume.

5. The process according to claim 1 for the manufacture of the compound PFMVE (perfluoro(methyl vinyl ether)) having the formula (I), wherein the fluorination (A) reaction is performed in a tube reactor system, in a continuous flow reactor system, in a coil reactor system, or in a microreactor system, preferably in a microreactor system, and wherein the fluorine (F₂) concentration in the F₂-fluorination gas is in a range of from about 85% by volume of elemental fluorine (F₂) up to about almost 100% by volume of elemental fluorine (F₂), more preferably of from about 90% by volume of elemental fluorine (F₂) up to about almost 100% by volume of elemental fluorine (F₂), based on the total F₂-fluorination gas composition as 100% by volume.

6. A process according to claim 1 for the manufacture of the compound PFMVE (perfluoro(methyl vinyl ether)) having the formula (I), wherein the direct fluorination reaction (A) and/or the HF-elimination reaction (B) is carried out in a (closed) column reactor.

7. A process according to claim 1 for the manufacture of the compound PFMVE (perfluoro(methyl vinyl ether)) having the formula (I), wherein the liquid reaction medium of the direct fluorination reaction (A) is circulated in a loop in a (closed) column reactor to perform the fluorination reaction (A), while the fluorination gas comprising elemental fluorine (F2) is fed into the (closed) column reactor and is passed through the liquid reaction medium to react with the compound HFE-254 (1,1,2,2-tetrafluoro-1-(methoxy)ethane) of formula (III); preferably wherein the loop is operated with a circulation velocity in the range of from about 1,000 l/h to about 2,000 l/h, more preferably in the range of from about 1,250 l/h to about 1,750 l/h; still more preferably wherein the loop is operated with a circulation velocity in the range of from about 1,500 l/h+200 l/h; even more preferably wherein the loop is operated with a circulation velocity in the range of from about 1,500 l/h+100 l/h; and most preferably wherein the loop is operated with a circulation velocity in the range of from about 1,500 l/h+50 l/h.

8. The process according to claim 7, wherein for the direct fluorination reaction (A) the closed column reactor is equipped with at least one of the following:
(i) at least one heat exchanger (system), at least one liquid reservoir, with inlet and outlet for, and containing the liquid reaction medium;
(ii) a pump for pumping and circulating the liquid reaction medium;
(iii) one or more nozzle jets, preferably wherein the one or more nozzle jets are placed at the top of the column reactor, for spraying the circulating reaction medium into the closed column reactor;
(iv) one or more feeding inlets for introducing the fluorination gas comprising or consisting of elemental fluorine (F2) into the (closed) column reactor;
(v) optionally one or more sieves, preferably two sieves, preferably the one or more sieves placed at the bottom of the (closed) column reactor;
(vi) and at least one gas outlet equipped with a pressure valve, and at least one outlet for withdrawing the fluorinated compound TFTFME (1,1,2,2-tetrafluoro-1-(trifluoromethoxy)ethane), (E 227), of formula (II) from the (closed) column reactor.

9. A process according to claim 1 for the manufacture of the compound PFMVE (perfluoro(methyl vinyl ether)) having the formula (I), wherein the liquid reaction medium of the HF-elimination reaction (B) is circulated in a loop in a (closed) column reactor to perform the HF-elimination reaction (B), and wherein the loop is operated with a circulation velocity in the range of from about 1,000 l/h to about 2,000 l/h, preferably in the range of from about 1,250 l/h to about 1,750 l/h; more preferably wherein the loop is operated with a circulation velocity in the range of from about 1,500 l/h+200 l/h; even more preferably wherein the loop is operated with a circulation velocity in the range of from about 1,500 l/h±100 l/h; and most preferably wherein the loop is operated with a circulation velocity in the range of from about 1,500 l/h+50 l/h.

10. The process according to claim 9, wherein for the HF-elimination reaction (B) the closed column reactor is equipped with at least one of the following:
(i) at least one heat exchanger system, at least one liquid reservoir, with inlet and outlet for, and containing the liquid reaction medium;
(ii) a pump for pumping and circulating the liquid reaction medium;
(iii) one or more nozzle jets, preferably wherein the one or more nozzle jets are placed at the top of the column reactor, for spraying the circulating reaction medium into the closed column reactor;
(iv) optionally, in case of (i) preferably performing the HF-elimination reaction as a(n) (exothermic) elimination reaction in the presence of one or more nitrogen-containing organic bases, one or more feeding inlets for introducing the one or more nitrogen-containing organic bases into the closed column reactor;
(v) optionally one or more sieves, preferably two sieves, preferably the one or more sieves placed at the bottom of the closed column reactor;
(vi) and at least one gas outlet equipped with a pressure valve, and at least one outlet for withdrawing the compound PFMVE (perfluoro(methyl vinyl ether)) having the formula (I) from the closed column reactor.

11. The process according to claim 6 for the manufacture of the compound PFMVE (perfluoro(methyl vinyl ether)) having the formula (I), wherein column reactor is a packed bed tower reactor, preferably a packed bed tower reactor is packed with fillers resistant to the reactants and especially resistant to elemental fluorine (F₂) and to hydrogen fluoride (HF) such as, e.g., with Raschig fillers, E-TFE fillers, and/or HF-resistant metal fillers, e.g., Hastelloy metal fillers, and/or (preferably) HDPTFE-fillers, more preferably wherein the packed bed tower reactor is a gas scrubber system (tower) which is packed with any of the before mentioned HF-resistant Hastelloy metal fillers and/or HDPTFE-fillers, and preferably with HDPTFE-fillers.

12. The process according to claim 1 for the manufacture of the compound PFMVE (perfluoro(methyl vinyl ether)) having the formula (I), wherein the direct fluorination reaction (A) and/or the HF-elimination reaction (B) is carried out in at least one step in a continuous flow reactor with upper lateral dimensions of about ≤5 mm, or of about ≤4 mm, more preferably in at least one step in a microreactor;
still more preferably wherein the direct fluorination reaction (A) and/or the HF-elimination reaction (B) is carried out in at least in one step as a continuous processes, wherein the continuous process is performed in at least one continuous flow reactor with upper lateral dimensions of about ≤5 mm, or of about ≤4 mm;
even more preferably wherein the direct fluorination reaction (A) and/or the HF-elimination reaction (B) is carried out in at least in one step as a continuous processes, wherein the continuous process is performed in at least one microreactor.

13. A process according to claim 1 for the manufacture of the compound PFMVE (perfluoro(methyl vinyl ether)) having the formula (I), characterized in that prior to starting any of the process steps (A) and (B) one or more of the reactors used, preferably each and any of the reactors used, are purged with an inert gas or a mixture of inert gases, preferably with He (helium) and/or N₂ (nitrogen) as the inert gas, more preferably with N₂ (nitrogen) as the inert gas.

14. A process according to claim 1 for the manufacture of the compound PFMVE (perfluoro(methyl vinyl ether)) having the formula (I), characterized in that in the fluorination reaction step (A) the reaction is performed in a SiC-reactor; preferably in that in the fluorination reaction step (A) the reaction is performed in a SiC-microreactor.

15. A process according to claim 1, for the manufacture of the compound PFMVE (perfluoro(methyl vinyl ether)) having the formula (I), characterized in that in the HF-elimination step (B) the reaction is performed in a nickel-reactor (Ni-reactor) or in a reactor with an inner surface with high nickel-content (Ni-content); preferably in that in the HF-elimination step (B) the reaction is performed in a nickel-microreactor (Ni-microreactor) or in a microreactor with an inner surface with high nickel-content (Ni-content).

16. A process according to claim 1 for the manufacture of the compound PFMVE (perfluoro(methyl vinyl ether)) having the formula (I), characterized in that, independently, the product yielding from fluorination reaction step (A) and/or the product yielding from HF-elimination step (B) are subjected to distillation.
